(19) United States
(12) Patent Application Publication
(10) Pub. No.: US 2007/0134233 A1
(43) Pub. Date: Jun. 14, 2007

(54) Compositions and methods relating to organ-, tissue-, and cell-specific antibodies

(75) Inventors: Wen Lin Seng, Westborough, MA (US); George Serbedzija, Sudbury, MA (US); Patricia McGrath, Cambridge, MA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Phylonix Pharmaceuticals, Inc., Cambridge, MA (US)

(21) Appl. No.: 11/048,122

(22) Filed: Jan. 31, 2005

Related U.S. Application Data
(60) Provisional application No. 60/540,666, filed on Jan. 30, 2004.

(51) Int. Cl. A61K 39/395; C07K 16/18

(52) U.S. Cl. 424/133.1; 530/387.1; 435/70.21

(57) ABSTRACT

Disclosed are methods for producing organ-, tissue-, or cell-specific antibodies, antibodies against developmentally regulated antigens, antibodies against disease-associated antigens, and antibodies against antigens that are modulated during a physiological response to an agent (e.g., in response to a drug). Also disclosed are methods for screening an agent for activity in modulating angiogenesis, as well as antibodies specific for angiogenic vessels.

### CROSS REFERENCES TO RELATED APPLICATIONS

The present application claims priority to U.S. Provisional Patent Application No. 60/540,666, filed Jan. 30, 2004, the entire disclosure of which is incorporated by reference herein.

### BACKGROUND OF THE INVENTION

I. Field of the Invention

The present invention resides in the field of production and identification of antibodies, particularly antibodies that are useful in the dissection of molecular pathways involved in, e.g., developmental- and/or disease-associated physiological responses. In addition, the present invention relates to the field of angiogenesis, particularly to methods of screening agents for activity in modulating angiogenesis, as well as compositions useful therein.

II. Description of Related Art

A. Molecular Targets of Development and Disease

Identification of molecular targets involved in developmental processes (e.g., tissue or organ morphogenesis) has implications for therapeutic intervention of disease. Various molecules involved in development have been shown to reappear during disease states (e.g., estrogen receptor expression in fetal prostate and in prostate cancer (Adams et al., Prostate 52:69-81, 2002); expression of Notch pathway proteins and PSA-NCAM during mammalian nervous system development and in multiple sclerosis (MS) lesions (John et al., Nat. Med. 8:1115-21, 2002; Reynolds et al., Brain 125:1972-9, 2002); expression of laminin β1 during metanephros development and membranous glomerulonephritis (Fisher et al., Nephrol. Dial. Transplant. 15:1956-64, 2000); expression of homeobox gene products during development and oncogenesis (Bodey et al., Anticancer Res. 20:2717-21, 2000); expression of various developmentally-regulated and cell cycle proteins during Alzheimer's Disease (Jordan-Sciutto and Bowser, Front Biosci. 15:D100-12, 1998; Hamre et al., Neurosci. Lett. 98:264-71, 1989); expression of the atrial isoform of alkali myosin light chain (MLC1A) during development of the heart and in different disease states of the human ventricle (Trahair et al., J. Mol. Cell Cardiol. 25:577-85, 1993)). Therefore, characterization of organ-or tissue-specific molecules expressed during organogenesis or histogenesis is a useful strategy for identifying molecules involved in disease states as well as for identifying targets for drug development.

Many of these important molecular markers associated with the development of tissues and organs have yet to be determined. For example, to date, only a few molecules involved in the morphogenesis of the digestive system have been identified (e.g., mammalian orthologs of foxA2, gata-6, hhx, and pdx-1 (see, e.g., Lai et al., Genes Dev. 5:416-27, 1991; Laverriere et al., J Biol. Chem. 269:23177-84, 1994; Martinez Barbera et al., Development 127:2433-45, 2000; Miller et al., EMBO J., 1994)). These genes are only a small fraction of the yet unidentified molecular targets that play important roles in the complex pathways of digestive system development and maintenance.

One example of a developmental process that has implications for disease is the formation of new blood vessels. New blood vessel formation can be categorized as either (a) vasculogenesis, which is the formation of blood vessels from precursor cells (e.g., angioblasts) (see Fouquet et al., Dev. Biol. 183:37-48, 1997) or (b) angiogenesis, which is the creation of new vessels from existing vessels. Angiogenic vessels form during normal tissue growth and repair in a series of sequential steps: an endothelial (EC) cell, which forms the wall of an existing small blood vessel, becomes activated, secretes enzymes that degrade the extracellular matrix, invades the matrix, and begins dividing. Eventually, strings of new endothelial cells organize into hollow tubes, creating new networks of blood vessels. Ordinarily, endothelial cells lie dormant. However, during, for example, tissue repair and pathological conditions (e.g., tumorigenesis, diabetic retinopathy (DR), and age-related macular degeneration (AMD)), short bursts of blood vessel growth occur in localized regions in tissues.

New capillary growth is tightly controlled by a finely tuned balance between factors that activate or inhibit endothelial cell growth. About 15 proteins are known to activate endothelial cell growth and movement, including, e.g., angiopoietins, epidermal growth factor (EGF), estrogen, fibroblast growth factors, prostaglandin, tumor necrosis factor (TNF), vascular endothelial growth factor (VEGF), and granulocyte stimulating factor (G-CSF). (See, e.g., Zetter, Ann. Rev. Med. 49:407-24, 1998.) Some of the known naturally occuring inhibitors of angiogenesis include thromospondin-1 and -2, angiopoietin-2, angiostatin, endostatin, interferons, interleukin-1, interleukin-12, retinoic acid, and tissue inhibitor of metalloproteinase 1, 2, and 3. (See Zetter, supra.) In the case of, for example, tumorigenesis, by blocking the development of new blood vessels, a tumor's supply of oxygen and nutrients can be cut off and continued growth and metastasis of the tumor arrested.

One model system for dissecting angiogenic pathways is the zebrafish. Development of blood vessels in zebrafish during embryogenesis is similar to the process in all vertebrates. During vasculogenesis, the dorsal aorta (DA) and posterior cardinal vein (PCV) are observed as early as 22 hours post fertilization (hpf). Later, new vessels sprout from these 2 major vessels during the process of angiogenesis. The major angiogenic vessels, including intersegmental vessels (ISVs) and head region vessels, begin sprouting 1.2-1.5 days post fertilization (dpf). (See Isogai et al., Developmental Biology 230:278-301, 2001.) Tissue angiogenic vessels have been observed at ˜4 dpf when the intestines and liver form.

At the molecular level, the processes of vasculogenesis and angiogenesis in zebrafish are also similar to other vertebrates. Several genes involved in these processes have been cloned in zebrafish and have been shown to be expressed in patterns similar to those in mammals. (See Liao et al., Development 124:381-89, 1997; Gehring et al., EMBO J. 17:4029-45, 1998; Lyons et al., Dev. Dynamics 212:133-140, 1998.) Molecular characterization of vessel formation has been used to identify a family of receptor tyrosine kinases (e.g., Flk-1, Flt-4, Tie-1, and Tie-2) that are specifically expressed on ECs and their progenitors. (See Hanahan, Science 277:48-50, 1997.) Flk-1 and Flt-1 are receptors for the vascular endothelial growth factor (VEGF), a potent endothelial mitogen. Specification of hemangioblasts from mesoderm requires expression of Flk-1 and the basic-helix-loop-helix transcription factor SCL/Tal (see Shalaby et al., Nature 376:62-66, 1995; Visvader et al., Genes Dev. 12:473-479, 1998). Subsequent differentiation of hemangioblasts into angioblasts is accompanied by expression of the receptors Flt-1 and Tie-1. (Dumont et al., Dev. Dynamics 203:80-92, 1995.) Differentiation and organization of ECs into tubes requires expression of the Tie-1 receptor. Formation of vessels also requires the recruitment of supporting cells such as, e.g., desmin-expressing pericytes (in the case of capillaries) and smooth muscle cells (in the case of larger vessels). Smooth muscles cells can selectively express α-smooth muscle actin.

As indicated above, angiogenesis is involved in the pathogenesis of various disease states. For example, angiogenesis has been shown to be involved in the development and spreading of tumors. Essentially all solid tumors are neovascularized by the time they are detected, and research has shown that tumor growth and metastasis are angiogenesis-dependent. (Folkman & Hannahan, Princess Takamatsu Symp. 22:339-47, 1991.) Angiogenesis is typically activated during multistage tumorigenesis prior to the emergence of solid tumors. Generally, tumorigenesis proceeds through at least two distinct preneoplastic stages. The first step involves a switch from quiescene to hyperproliferation of oncogenic cells. The second step involves induction of angiogenesis, whereby the normally quiescent vasculature is activated to proliferate and form new capillaries. (See, e.g., Parangi et al., Proc. Natl. Acad. Sci. USA 93:2002-7, 1996) When enough tumor cells become angiogenic so as to induce neovascularization, thereby supplying the tumor cells with oxygen and nutrients, the tumor can expand progressively and mestastasize (Folkman & Hannahan, supra). This “angiogenic switch” is an important step toward malignancy.

Several molecular components of angiogenic pathways have been identified, (see supra) and have been shown to be involved in tumorigenesis. For example, VEGF is known to induce angiogenesis in tumors, while thromobspondin-1 (TSP-1) is a negative regulator of tumor angiogenesis. (Zhang et al., Proc. Natl. Acad. Sci. USA 100:12718-23, 2003.) Other proteins implicated in tumor angiogenesis include, e.g., hepatocyte growth factor/scatter factor (HGF/SF), TGF-β, macrophage migration inhibitory factor (MIF), and COX-2. (See Zhange et al., supra; Goumans et al., Trends Cardiovasc. Med. 13:301-7, 2003; Nishihira et al., Ann. N.Y. Acad. Sci. 995:171-82, 2003; Gately & Kerbel, Prog. Exp. Tumor Res. 37:179-92, 2003.)

Prevention of blood vessel development has been shown to be a mechanism of action of many successful chemopreventive drugs. Further, it has been suggested that anti-angiogenic drugs can be used for chemoprevention in higher risk populations or in early intervention. (See Bisacchi et al., Cancer Detect Prev. 27:229-38, 2003.) However, current chemopreventive drugs suffer from the several disadvantages, including, e.g., various toxic side effects that substantially limit their therapeutic window. Therefore, there is a need in the art for the development of novel and safe angiogenesis inhibitors, as well as a need for methods of identifying such inhibitors.

New blood vessel formation is also involved in the pathogenesis of many ocular diseases. For example, diabetic retinopathy (DR) and age-related macular degeneration (AMD), the two leading causes of blindness in adults in the industrialized world, both involve vascular abnormalities, proliferation, and leakage of new blood vessels. Retinopathy of prematurity (ROP) is a major cause of newborn blindness in premature infants maintained by oxygen supplementation during the postnatal period. This disease involves intense neovascularization of the retina and leads to retinal detachment. Another cause of blindness is corneal neovascularization (CNV), which often results from injury and infection in the cornea. Current surgical, laser photocoagulation, and photodynamic therapies do not always result in improved vision and exhibit high risk of side effects. Control of malformation of the new blood vessels in eye is considered as an alternative and less invasive therapy.

Hypoxia is believed to be a major mechanism for abnormal neovascularization in eye diseases (Semenza, Genes & Development 14:1983-1991, 2000; Schwesinger et al., Am. J. Path. 158:1161-72,2001; Smith, Acta Paediatr. Supplement 91:26-8,2002; Shih et al., J. Clin. Invest. 112:50-57, 2003). Several proteins are now known to play significant roles in the oxygen homeostasis pathway. For example, hypoxia inducible factor-1 (HIF-1), a transcription factor comprised of two subunits, HIF-1α and HIF-1β, binds to the hypoxia-response element located at the promoter region of certain genes, thus up-regulating the expression of these genes. Several genes are known to respond to HIF-1; these genes include, for example, several glucose/energy metabolism genes, which are associated with oxygen homeostasis. In addition, several vascular development/remodeling genes have also been shown to respond to HIF-1, including, e.g., VEGF, nitric oxide (NO) synthase 2, Flt-1, and IGF. In addition, VEGF has been shown to be present in corneal stroma after corneal injury and hypoxia (Moromizato et al., Am. J. Path. 157:1277-81, 2000). VEGF also plays a prominent role in the progression of age-related macular degeneration (ARMD) and diabetic retinopathy (DR); high levels of VEGF have been documented in the vitreous of human retinopathies in macular degeneration (MD) patients (Rasmussen et al., Human Gene Therapy 12:2029-32, 2001). Increasing evidence indicates that additional angiogenic factors and inhibitors also play pivotal roles in pathological angiogenesis. (See, e.g., Bouck, Trends Mol. Med. 8:330-4, 2002).

Current methods for identifying molecular targets of development and disease, including, for example, targets involved in angiogenesis, suffer from several disadvantages. Some current methods for dissecting molecular pathways involved in organogenesis or histogenesis include genetic screens or anti-sense technology, which are time consuming and costly. Similarly, while monoclonal antibodies (mAbs) are widely used as research, therapeutic, diagnostic, and imaging reagents, these reagents are not typically used for dissecting complex molecular pathways due to, e.g., time and cost restraints associated with their production. For example, conventional methods of monoclonal antibody production typically take as long as 3-9 months and in excess of $10,000 per antibody. While an alternative approach for generating mAbs using a multiple site immunization strategy allows for production of mAbs in less than one month (see Kilpatrick et al., Hybridoma 16:381-9, 1997; Wring et al., J. Pharm. Biomed. Anal. 19:695-707, 1999), current methods for screening such antibodies do not allow the efficient identification of antibodies to tissue- or organ-specific antigens, including such antigens that are developmentally regulated. Current methods for screening antibodies require, for example, either that the target antigen be previously identified or isolated (e.g., such as in an ELISA format) or screening of tissues in situ using laborious tissue sectioning procedures (see, e.g., Meyer et al., J. Neurosci. 7:512-21, 1987).

In addition, conventional methods do not allow for efficient identification of molecular targets in their native physiological context, nor do current methods allow for effective and efficient screening of drug candidates in a complex physiological environment. Current assays for morphogenic events are generally divided into three categories: in vitro, ex vivo, and in vivo assays. For example, in the case of angiogenesis, the following assays are conventionally utilized: (1) for in vitro assays, human vascular endothelial cells (e.g., HUVEC or HDMEC) are used for EC proliferation, cord formation, and EC migration assays; (2) for ex vivo assays, sections of freshly harvested aorta or artery vessels from different animal models are kept in culture plates and endothelial cells sprouting from these vessel sections are assessed; and (3) for in vivo assays, vessel formation is induced and assessed, e.g., either in rat cornea, chick choriallantoin membrane, or matrigel plug in mice. Typically, in in vitro and ex vivo assays, different morphogenic events (e.g., cell differentiation, proliferation, migration, tissue or organ formation (e.g., cord formation in angiogenesis)) can be clearly defined, although not under complex physiological conditions. Conversely, for in vivo assays, morphogenic effects can be assessed in complex physiological conditions; however, this approach is time consuming and expensive and effects on different types of developmental events (e.g., differentiation, proliferation, migration, tissue or organ formation) cannot be readily distinguished. Accordingly, in vivo assays are not typically used as a primary screen for identification of molecular targets or lead compounds for drug discovery.

For example, one assay that allows for direct observation of a developmental process in vivo is the chick embryo chorioallantoic membrane (CAM) assay for angiogenesis. This time consuming and expensive assay involves the opening of a small window in the shell of a chicken egg so as to permit observation of the developing CAM, including blood vessel formation. Substances (e.g., isolated cell populations or agents) can be tested for angiogenic effects by exposing the CAM to the test substance and then measuring the angiogenic response. However, while angiogenesis can be measured in the membrane surrounding the chick embryo using this assay, angiogenic or other responses cannot be measured in other tissues of the chick embryo itself, nor can drug effect in endothelial cell proliferation, sprouting, or migration be distinguished

Further, with respect to monoclonal antibodies, the use of mAbs in specific in vivo assays is hindered by the limited cross-reactivity of mAbs between animals of different classes or phyla. Model systems for developmental biologists include, for example, mouse, zebrafish, Drosophila, and C. elegans. Despite differences of organ and tissue morphogenesis, conserved molecular programs regulate development in animals of evolutionary divergent classes, including, e.g., mammals, fish, and insects. However, antibodies generated against mammalian antigens, for example, seldom cross-react with proteins of non-mammalian species, making use of these mAbs for in vivo assays impractical. Moreover, most available mAbs target multiple tissues or organs and, therefore, cannot be used for tissue- or organ-specific screening assays.

Therefore, there is a need in the art for specific reagents and more efficient methods for the identification of molecular targets of development and disease, particularly the identification of these targets in a physiological context. Such reagents and methods would be useful for the identification of novel molecular pathways and their components for drug target validation, as well as for the dissection of these pathways in vivo. Further, there is a need for assay systems that facilitate more rapid and efficient identification of agents that modulate these development- and disease-associated pathways by allowing effects of agents to be clearly defined in a complex physiological environment. The present invention as described herein satisfies these needs and more.

### BRIEF SUMMARY OF THE INVENTION

In one aspect, the present invention provides methods for producing an organ-, tissue-, or cell-specific antibody, the method comprising the following steps: (1) immunizing a mammal with cellular or extracellular material isolated from an animal of a species that is transparent through multiple stages of development to generate cells that secrete antibodies, wherein the cellular or extracellular material comprises antigenic determinants from a specific organ, tissue, or cell population; (2) contacting the antibodies with an intact animal of the transparent species, whereby the antibodies permeate the animal or portion thereof; and (3) detecting the antibodies within the animal and thereby identifying an antibody that binds to the specific organ, tissue, or cell population. In certain embodiments, the antigen recognized by the organ-, tissue-, or cell-specific antibody is developmentally regulated. The specific organ or tissue can be, for example, a blood vessel, liver, pancreas, pharynx, esophagus, pronephros, brain, heart, gut, or muscle. Suitable transparent animals include, for example, teleosts (e.g., zebrafish).

In typical variations, the mammal for immunization is a rodent (e.g., a mouse). In addition, suitable methods of immunization include, for example, performing multiple immunizations at sites proximal to a draining lymph node (e.g., RIMMS).

Further, in certain embodiments, the methods also include isolating the cellular or extracellular material. One particularly suitable method for isolating the cellular or extracellular material laser capture microdissection (LCM). In one specific embodiment, the cellular or extracellular material is isolated from an animal that has a disease or has been treated with a drug; in such embodiments, the intact animal of the transparent species can also have the disease or have been treated with the drug.

In a related aspect, the present invention provides methods of producing an antibody to a developmentally regulated antigen, the method comprising the following steps: (1) immunizing a mammal with cellular or extracellular material from an animal of a species that is transparent through multiple stages of development to generate cells that secrete antibodies; (2) contacting samples of one of the antibodies with a plurality of intact animals of the transparent species, said intact animals representing at least two different stages of development, whereby the antibody permeates the intact animals; (3) detecting the amounts and locations of the antibody specifically bound within the animals; and (4) determining whether a difference in the amount and/or location of bound antibody is present between animals in different developmental stages, whereby if a difference in the amount and/or location of bound antibody is present, the antibody is identified as specifically binding to a developmentally regulated antigen. Suitable mammals for immunization include, for example, rodents (e.g., mice). In certain embodiments, the methods further comprise isolating the cellular or extracellular material. In some variations of the methods, the developmentally regulated antigen is organ-, tissue-, or cell-specific.

In yet another aspect, the present invention provides monoclonal antibodies specific for vascular endothelial cells in zebrafish. Also provided is a kit for detecting vascular endothelial cells in zebrafish comprising a vascular endothelial cell-specific antibody as described herein. The certain embodiments, the kit includes a blocking reagent, a washing reagent, and/or a reagent capable of detecting the presence of the antibody.

The present invention also provides methods of determining whether an agent modulates angiogenesis, the method comprising the following steps: (1) contacting the agent with a zebrafish; (2) contacting the zebrafish with a monoclonal antibody that specifically recognizes vascular endothelial cells in zebrafish; (3) detecting the monoclonal antibody in the zebrafish to visualize an angiogenic process; and (4) comparing the angiogenic process in the zebrafish contacted with the agent with that in a control zebrafish not contacted with the agent to determine whether the agent modulates angiogenesis. The methods can further include (a) contacting a second zebrafish with the agent and (b) monitoring the second zebrafish for a response to the agent that is indicative of toxicity. In addition, the methods optionally include contacting the zebrafish with an agent that induces an angiogenic process.

In certain embodiments of the method of determining whether an agent modulates angiogenesis, the angiogenic process is migration or proliferation of vascular endothelial cells. In yet other embodiments, the angiogenic process is associated with ocular neovascularization.

In still another aspect of the present invention, methods for identifying a protein for drug target validation are provided, the methods comprising the following steps: (1) immunizing a mammal with cellular or extracellular material from an animal of a species that is transparent through multiple stages of development to generate cells that secrete antibodies, wherein the cellular or extracellular material comprises antigenic determinants from a specific organ, tissue, or cell population; (2) contacting the antibodies with an intact animal of the transparent species, whereby the antibodies permeate the animal or portion thereof; (3) detecting the antibodies within the animal and thereby identifying an antibody that binds to the specific organ, tissue, or cell population; (4) isolating an antigen specifically bound by the antibody; and (5) determining one or more biochemical characteristics of the isolated antigen to identify the protein for drug target validation. Suitable mammals for immunization include, for example, rodents (e.g., mice).

In certain variations of the method for drug target validation, the biochemical characteristic is an amino acid sequence for the antigen or a fragment thereof or, alternatively, a mass spectral fingerprint (e.g., a MALDI-TOF mass spectral fingerprint). In other embodiments, the methods further include identifying the gene that encodes the protein, or identifying a species variant of the protein or of the gene encoding the protein.

### BRIEF DESCRIPTION OF THE DRAWINGS

FIG. 1 depicts immunofluorescent staining of zebrafish with zebrafish organ-specific mAbs targeting liver.

FIG. 2 depicts a 2 dpf embryo stained by Phy-V002. Individual cells forming the eye vessels (A) and assembling to form the sub-intestinal vessels on the surface of the yolk (B and C) are shown at higher magnification (10× objective lens).

FIGS. 3A and 3B depict zebrafish vessels detected by microangiography and Phy-V001 staining. Normal 3 dpf embryos were fixed, permeabilized, and blocked, as described in whole mount immunostaining. FIG. 3A shows an embryo first incubated with Phy-V001 and then stained with rhodamine-conjugated secondary antibody. FIG. 3B shows a live 3 dpf embryo injected with rhodamine-conjugated microspheres. Phy-V001 did not stain the vasculogenic vessels, and no background staining was observed. Microangiography showed intense fluorescence in the heart chamber, DA, PCV, and ISVs. DA: dorsal aorta, H: heart, HV: head region vessels, ISV: intersegmental vessels, PCV: posterior cardinal vein, SIV: subintestinal vessels.

FIGS. 5A and 5B depict immunostaining with Phy-V001. FIG. 5A shows a whole mount immunostained as described in Example 5. White arrowheads indicate the site that the PAV bud from ISVs (40×). FIG. 5B shows immunohistochemistry of embryo sagital sections. Left panel: fluorescent images of Phy-V001 staining; long white arrows indicate strong staining by Phy-V001 in an area in the dorsal aorta (D); large white arrowheads indicate ISVs; the branching site of the ISVs in the dorsal aorta is strongly stained by Phy-V001; 1, 1.5, and 2 dpf (100×), 3 dpf (200×). Right panel: phase contrast of the same sections showing the dorsal aorta (D) and notocord (N). CV: caudal vein, ISV: intersegmental vessels.

FIG. 6 depicts endothelial cells (EC) stained by Phy-V001 mAb. 40hpf embryos were stained by Phy-V001 using whole mount immunostaining. Left panel shows the head region with several forming vessels (long white arrows) (100×). The right panel shows the ISVs in the trunk region (200×). A, B, C and D in the 2 middle panels are 200× magnification of the regions marked by white squares in the left and right panels. At this higher magnification, individual cells were identifiable with the strongest staining in the adhesion, advancing (migration), and budding (proliferation) regions of the cells.

FIGS. 7A and 7B depict angiogenic drug-treated embryos. 1 dpf embryos were treated with various concentrations of SU5416 (FIG. 16A) and flavopiridol (FIG. 16B). Whole embryo images were taken at 2 dpf stage at 40×, and confocal images of ISVs images were taken at 3 dpf stage at 100×, which were compiled from 20 slices of images using Photoshop 6.0.

FIGS. 8A and 8B depict establishment of quantitative in vivo microplate assay conditions using Phy-V001 mAb. Normal 1-6 dpf embryos were processed, and incubated with QuantaBlu for various times, as described in Example 5, infra (FIG. 8A). QuantaBlu incubation time was set at 60 min, and level of Phy-V001 staining in different age embryos was measured as RFU (FIG. 8B). Each point represents mean +S.D. (n=36).

FIGS. 9A-9D depict small molecule drug-treated zebrafish embryos: SU5416 (FIG. 9A), flavopiridol (FIG. 9B), COL-1 (FIG. 9C), COL-3 (FIG. 9D). 1 dpf embryos were treated with varying concentrations of each drug. At 2 dpf (blue line) and 3 dpf (red line), embryos were fixed and processed for the quantitative in vivo microplate assay, as described in Example 5, infra. QuantaBlu incubation time was set at 60 minutes for each sample. Inhibition of angiogenic vessel growth was calculated following formula (a) (see Example 5). Each point represented mean +S.E. (n=36). X axis represents the concentration of each drug. Y axis represents the % inhibition of angiogenic vessel development compared to controls.

FIG. 10 depicts the screening strategy for the quantitative in vivo zebrafish angiogenesis bioassay.

FIG. 12 depicts whole mount immunostaining with Phy-V002. ˜48 hpf embryos were stained by Phy-V002 following the whole mount immunostaining technique described in Example 9, supra. Higher magnification (10× object lens) image were taken for regions corresponding to A, B, and C in the whole embryo image (4× object lens). A: cranial vessels including eye vessels; B: subintestinal vessels; and C: tail vessels. Individual endothelial cells can be clearly seen in the forming vessels.

FIG. 13 depicts the linear relationship of Phy-V002 staining signal with the number of embryos. Whole animal ELISA was performed in 2 dpf embryos. X axis represents the number of embryos per well, Y axis represents the random fluorescence unit signal from the end-product of the reporter enzyme. Each point represents mean +SD (n =6)

FIGS. 14A and 14B depict dose-response curves of 2-ME and flavopiridol. ˜20 hpf embryos were treated with 2-ME (FIG. 14A) and flavopiridol (FIG. 14B) for 28 hours. Control and drug treated embryos were processed for whole animal Phy-V002 ELISA. % inhibition in EC proliferation was plotted against drug concentrations. Each point represents mean +SE (n=24).

FIGS. 15A and 15B depict whole mount Phy-V002 staining in control and tetracycline treated embryos. ˜20 hpf embryos were treated with 0.2 mM NaOH (Control) (FIG. 15A) and 600 μM tetracycline (FIG. 15B). Drug treatments were stopped at ˜48 hpf, embryos were fixed and processed for whole mount Phy-V002 staining. The long arrows in FIG. 15A indicate Phy-V002 stained cells lined up at the intersection of tail vein and intersegmental vessels (ISVs). The short arrow in FIG. 15B pointed at the site of missing ISVs, and Phy-V002 stained cells aggregate was observed. The tail veins in tetracycline treated embryos were abnormal compared to control.

FIG. 16 depicts anti-angiogenic effect of SU5416. Control (0.1% DMSO and 0.2% ethanol) and SU5416 treated 2 dpf embryos were stained with Phy-V002, followed by staining with rhodamine-conjugated secondary antibody. The intact cranial vessels (CV) and intersegmental vessels (ISV) in the control embryos are indicated by arrows. The vessel patterns of 1 μM treated embryo were abnormal when compared to control embryos (stars). As the concentration increased, the angiogenic vessels were nearly completely absent.

FIG. 17 depicts morphometric analysis of in vivo vessel formation. Higher magnification (10×) images of the same embryos from FIG. 18 were used for morphometric analysis. Row (A) shows the region chosen for vessel analysis, row (B) shows the area (tail ISV) of each section used for morphmetric analysis, row (C) shows the threshold images of row (B), and row (D) are the results of morphometric analysis expressed in both absolute numbers and percentage compared to control.

FIG. 18 depicts a dose response curve of inhibition of in vivo vessel formation by SU5416. The results from FIG. 19 were used to calculate values for % inhibition, which were then plotted against drug concentration.

### DETAILED DESCRIPTION OF THE INVENTION

I. Definitions

Unless defined otherwise, all technical and scientific terms used herein have the same meaning as commonly understood by those of ordinary skill in the art to which this invention belongs. The following references provide one of skill with a general definition of many of the terms used in this invention: Singleton et al., Dictionary of Microbiology and Molecular Biology (2d ed. 1994); The Cambridge Dictionary of Science and Technology (Walker, ed., 1988); and Hale & Marham, The Harper Collins Dictionary of Biology (1991). As used herein, the following terms and phrases have the meanings ascribed to them unless specified otherwise.

The terms “a,” “an,” and “the” include plural referents unless the context clearly indicates otherwise.

The term “agent” as used herein includes any element, compound, or entity, including, but not limited to, e.g., pharmaceutical, therapeutic, pharmacologic, environmental or agricultural pollutant or compound, aquatic pollutant, cosmeceutical, drug, toxin, natural product, synthetic compound, or chemical compound.

The term “natural compound” as used herein includes a molecule isolated, extracted, or purified from a plant, animal, yeast, bacterium, or other microorganism. A natural compound includes, e.g., among other things, organic molecules belonging to the broad biochemical classes of peptides, proteins, glycoproteins, nucleic acids, carbohydrates, lipids, fats, glycolipids, as well as more complex molecules which comprise, e.g., elements of more than one of these basic biochemical classes.

The term “synthetic compound” as used herein includes a molecule synthesized de novo or produced by modifying or derivatizing a natural compound.

The term “subject” as used herein means an animal. The term “animal” as used herein includes a vertebrate animal such as, e.g., a vertebrate fish. Vertebrate fish include teleosts, such as, e.g., zebrafish, medaka, Giant rerio, and puffer fish. The term “teleost” as used herein means of or belonging to the Teleostei or Teleostomi, a group consisting of numerous fishes having bony skeletons and rayed fins. Teleosts include, for example, zebrafish, medaka, Giant rerio, and puffer fish.

The term “cellular or extracellular material” as used herein means biological material isolated from an animal that includes molecular components of animal cells (including, e.g., cytosolic or membrane bound components) and/or molecular components of associated extracellular material (e.g., extracellular matrix). In certain embodiments of the methods described herein, cellular or extracellular material includes an organ (or region thereof), a tissue (or region thereof), or a population of cells (or sub-population thereof) of an animal, or any sub-set of molecular components derived therefrom.

The term “tissue” as used herein means a group of similar cells and associated intercellular substances organized into a structural and functional unit. For example, tissues generally include, e.g., vascular, epithelial, lymphatic (including, e.g., gut-associated or muscosa-associated), skeletal muscle, smooth muscle, central nervous, peripheral nervous, glial, bone, cardiac, renal, adipose, connective, hematopoietic, or myeloid. The term can also refer to a tissue associated with a particular organ (e.g., brain, gut tissue, liver, pancreas, eye, and the like).

The term “organ” as used herein means an animal body structure composed of multiple tissues grouped together in a structural and functional unit. Organs include, for example, brain, spleen, heart, kidney, lung, pancreas, pharynx, esophagus, intestine (gut), gall bladder, lymph nodes, vessels of the circulatory system, eyes, peripheral or central nerves, and the like.

The term “antigen” as used herein means any molecule that is capable of binding to the antigen binding site of an antibody. “Target antigen” as used herein means an antigen that binds to the antigen binding site of a predetermined antibody. “Antigenic determinant” as used herein means any molecule or region thereof (e.g., an amino acid motif of a polypeptide) that is capable of eliciting an immune response, either alone or when conjugated to a carrier molecule (such as, e.g., keyhole limpet hemacyanin (KLH), bovine serum albumin (BSA), or ovalbumin).

In reference to an antigen, the term “cell-specific” as used herein means that the antigen is expressed differentially within or on at least one cell type of an animal during at least one developmental stage, including, for example, embryonic, larval, or adult stages. In the context of antigen expression within or on a cell, “differential expression” means that the degree of expression in the particular tissue as compared with other animal tissues is sufficient to allow visualization of the cell when the antigen is specifically detected (e.g., using a labeled monoclonal antibody that specifically binds the cell-specific antigen). Thus, for example, an antigen is cell-specific if the antigen is expressed exclusively in one cell type (i.e., without detectable expression in any other animal cells or tissues). In addition, for example, an antigen is tissue-specific if the antigen is expressed in one (“first”) cell type of an animal as well as in other cells or tissues if the expression in the first cell type is sufficiently greater than in surrounding cells or tissues so as to allow the first cell type to be distinguished visually from the surrounding tissues when the antigen is detected. Also, for example, an antigen is cell-specific if the antigen is expressed in more than one cell type or tissues of an animal, but not all cells or tissues, and detection of the antigen allows independent visualization of one or more cell types expressing the antigen, such that the one or more cell types can be distinguished visually from each other. As used herein, “cell-specific antibody” refers to an antibody that specifically binds to a cell-specific antigen.

In reference to an antigen, the term “tissue-specific” as used herein means that the antigen is expressed differentially in at least one tissue of an animal during at least one developmental stage, including, for example, embryonic, larval, or adult stages. In the context of antigen expression in a tissue, “differential expression” means that the degree of expression in the particular tissue as compared with other animal tissues is sufficient to allow visualization of the tissue when the antigen is specifically detected (e.g., using a labeled monoclonal antibody that specifically binds the tissue-specific antigen). Thus, for example, an antigen is tissue-specific if the antigen is expressed exclusively in one tissue (i.e., without detectable expression in any other animal tissues). In addition, for example, an antigen is tissue-specific if the antigen is expressed in one (“first”) tissue of an animal as well as in other tissues if the expression in the first tissue is sufficiently greater than in surrounding tissues so as to allow the first tissue to be distinguished visually from the surrounding tissues when the antigen is detected. Also, for example, an antigen is tissue-specific if the antigen is expressed in more than one tissue of an animal, but not all tissues, and detection of the antigen allows independent visualization of one or more tissues expressing the antigen, such that the one or more tissues can be distinguished visually from each other. As used herein, “tissue-specific antibody” refers to an antibody that specifically binds to a tissue-specific antigen.

In reference to an antigen, the term “organ-specific” as used herein means that the antigen is expressed differentially in at least one organ of an animal during at least one developmental stage, including, for example, embryonic, larval, or adult stages. In the context of antigen expression in an organ, “differential expression” means that the degree of expression in the particular organ as compared with other animal tissues or organs is sufficient to allow visualization of the organ when the antigen is specifically detected (e.g., using a labeled monoclonal antibody that specifically binds the organ-specific antigen). Thus, for example, an antigen is organ-specific if the antigen is expressed exclusively in one organ (i.e., without detectable expression in any other animal tissues or organs). In addition, for example, an antigen is organ-specific if the antigen is expressed in one (“first”) organ of an animal as well as in other tissues or organs if the expression in the first organ is sufficiently greater than in surrounding tissues or organs so as to allow the first organ to be distinguished visually from the surrounding tissues or organs when the antigen is detected. Also, for example, an antigen is organ-specific if the antigen is expressed in more than one organ of an animal, but not all organs, and detection of the antigen allows independent visualization of one or more organs expressing the antigen, such that the one or more organs can be distinguished visually from one another. As used herein, “organ-specific antibody” refers to an antibody that specifically binds to an organ-specific antigen.

The term “intact” in reference to an animal means an animal specimen having substantially all native organs and tissues, where the organs and tissues retain their native structural organization in the animal. “Substantially all native organs and tissues” means at least about 80%, preferably at least about 90%, more preferably at least about 95%, and most preferably about 100% of the animal's native organs and tissues.

An “intact portion” in reference to an animal means an animal specimen having at least about 5%, typically at least about 10%, more typically at least about 20%, even more typically at least about 30%, preferably at least about 40%, and more preferably at least about 50%, but less than substantially all, native organs and tissues, where the organs and tissues retain their native structural organization in the respective animal portion. In the context of antibody screening, an intact portion will typically have at least one intact organ (e.g., an organ for which antibody specificity is desired), typically at least one intact organ and one or more other tissues or organs, and most typically at least two or more intact organs. Intact portions can be generated, for example, by sectioning the transparent animal cross, longitudinally, or sagitally. For example, where an portion of the transparent animal is generated by taking a cross-section, an intact portion will have at least about 5%, typically at least about 10%, more typically at least about 20%, even more typically at least about 30%, preferably at least about 40%, and more preferably at least about 50% of organs and tissues as determined by length of the section as compared to the length of the whole intact animal. In certain embodiments, an intact animal portion can include, e.g., an animal's head, torso, or any appendage(s), or any combination thereof, where each head, torso, appendage(s), or combination thereof retains the native structural organization of the respective organs and tissues. Thus, in the case of an intact vertebrate animal portion comprising an animal's head, the head would typically include, for example, the animal's brain; eyes; skull; olfactory organs; blood vessels, muscles, and glands native to the head; and peripheral neural networks innervating, e.g., eyes, muscles, and glands.

The term “transparent” in reference to an animal means that a substantial proportion of the animal's tissues or organs are observable within the intact animal, either with the naked eye or by light microscope and either with or without labeling or staining procedures (e.g., immunofluorescence). “Substantial proportion of tissues or organs” means that at least about 30%, typically at least about 40%, more typically at least about 50%, preferably at least about 60%, more preferably at least about 70%, and most preferably at least about 80% or at least about 90% of the animal's tissues or organs.

In reference to an animal, including a particular tissue, organ, or system of the animal, the term “development” as used herein refers to the process of natural progression in physical maturation from a previous, lower, or embryonic stage to a later, more complex, or adult stage.

In reference to an antigen, the term “developmentally regulated” as used herein means that the antigen is expressed differentially through at least two stages of development. “Differential expression through at least two stages of development” means that, when the antigen is detected in the animal at multiple developmental stages, the amount (degree of antigen expression) and/or location(s) of the antigen in an animal from one stage is significantly different from that in an animal of a second, different developmental stage. Accordingly, an “amount of antibody specifically bound” (with reference to the identification of an antibody that recognizes a developmentally regulated antigen) generally does not refer to a determination of an absolute quantity, but relative quantities of bound antibody between samples from different developmental stages (e.g., as determined by measuring relative intensity of signal from a detection reagent such as, for example, a fluorescently-labeled secondary antibody).

The term “morphogenic change” or “morphogenesis” as used herein refers to a change in the architectural features of an organism occurring during development, including, e.g., the proliferation of cells, the migration of cells, the differentiation of cells or tissues, or the formation of tissues or organs.

As used herein, in the context of an agent, the phrase “activity in modulating angiogenesis” or “angiogenic activity” refers to the ability of the agent to enhance, inhibit, or prevent one or more angiogenic processes. The term “angiogenic processes” refers to any morphogenic change associated with the angiogenesis, including, for example, endothelial cell proliferation or migration and formation or outgrowth of blood vessels or lymph vessels. In specific embodiments, an agent with angiogeneic activity is “anti-angiogenic,” which is defined herein as the ability of the agent to inhibit, prevent, or greatly reduce one or more angiogenic processes.

The term “disease” or “disease state” as used herein, in reference to an animal or any organ, tissue, or population of cells thereof, means a disorder of normal function such as, for example, a disorder typically associated with pathogenesis of a known disease (e.g., physiological states associated with cancer, inflammatory diseases, autoimmunity, neurodegenerative diseases, or metabolic diseases (e.g., liver disease or diabetes). A disease state can include, for example, hyperproliferation or hypoproliferation of cells (e.g., increased cell proliferation associated with tumorigenesis or increased proliferation of leukocytes associated with inappropriate immune or inflammatory responses); abnormal cellular differentiation (e.g., differentiation of cells to a cell-type not typically associated with a particular tissue or developmental stage; cell death (e.g., by apoptosis); over- or under-expression of proteins that regulate normal physiological responses (e.g., cell surface molecules or cytokines); abnormal cell migration; abnormal metabolic responses (e.g., increased lipid content in hepatocytes); or abnormal organ or tissue formation such as, for example, formation of blood vessels in a tissue and/or during a developmental stage not typically associated with new blood vessel formation (e.g., the new blood vessel formation by angiogenesis observed under hypoxic conditions).

The term “label” as used herein means any agent that (1) is or is intended to be joined, either covalently or non-covalently, to a molecule such as, e.g., an antibody, and (2) includes a detectable physical or chemical property. For example, known labels include, e.g., radionuclides, enzymes, enzyme substrates, enzyme cofactors, enzyme inhibitors, ligands (e.g., haptens), fluorophores, chromophores, luminescers, and magnetic particles. A label can be direct or indirect. An “indirect label” is a specifically bindable molecule (a “ligand”) that is detected using a labeled secondary agent (a “ligand binding partner”) that specifically binds to the indirect label (e.g., haptens, biotin, and the like). A “direct label” is detected without a ligand binding partner interaction (e.g., fluorophores, chromophores, luminescers, enzymes, enzyme cofactors, and the like).

The term “biochemical characteristic” in reference to an antigen means any detectable chemical, molecular, or physical characteristic of the antigen, typically determined using known biochemical analytical techniques. “Biochemical characteristics” can include, for example, molecular weight (e.g., as determined by mobility on SDS-PAGE or mass spectrometry), immunological cross-reactivity with an antibody (as determined, e.g., by ELISA or Western blot analysis), time of elution from a column under a defined set of chromatographic conditions (e.g., column type, buffer pH, buffer ionic strength, temperature, buffer hydrophobicity, flow rate, and the like, as determined using, e.g., HPLC), glycosylation, ability to specifically bind another molecule (e.g., as determined by immunoprecipitation of BIAcore analysis), amino acid sequence (determined by, e.g., Edman degradation), or pattern of peptides generated by one or more cleaving agents (e.g., an enzyme), as determined by, for example, mass spectrometry (e.g., MALDI-TOF MS). The term “gene” is used broadly to refer to any segment of DNA associated with a biological function. Thus, genes include coding sequences and/or the regulatory sequences required for their expression. Genes also include non-expressed DNA segments that, for example, form recognition sequences for other proteins. The term “nucleic acid” refers to a deoxyribonucleotide or ribonucleotide or polymer thereof that is in either single- or double-stranded form. Unless specifically limited, the term encompasses nucleic acids containing known analogues (synthenic and naturally occurring) of nucleotides, where the nucleotide analogues are metabolized in a manner similar to the reference nucleotides and the analogue-containing nucleic acids have similar binding properties as the reference nucleic acid. The term nucleic acid is used interchangeably with gene, cDNA, and mRNA encoded by a gene.

The term “isolated nucleic acid” means a single- or double-stranded nucleic acid (e.g., an RNA, DNA, or mixed polymer) that is substantially separated from other genome DNA sequences as well as proteins or complexes that naturally accompany a native sequence (e.g., ribosomes and polymerases). The term embraces a nucleic acid that has been removed from its naturally occurring environment as well as recombinant or cloned DNA isolates and chemically synthesized analogues or analogues biologically synthesized by heterologous systems. A substantially pure molecule includes isolated forms of the molecule. An “isolated polypeptide” or protein carries a similar meaning with the polypeptide or protein being substantially separated from any cellular contaminants and components naturally associated with the protein in vivo.

The terms “polypeptide,” “peptide” and “protein” are used interchangeably herein to refer to a polymer of amino acid residues. The terms apply to amino acid polymers in which one or more amino acid residue is an artificial chemical analogue of a corresponding naturally occurring amino acid, as well as to naturally occurring amino acid polymers.

II. Organ-, Tissue-, and Developmentally Regulated Antigen-specific Antibodies

The present invention provides methods for generating antibodies that specifically bind organ-, tissue-, or cell-specific antigens as well as methods for generating antibodies specific for developmentally regulated antigens. Generally, identification of the antibodies includes the utilization of intact transparent animals, or intact portions thereof, for screening multiple antibodies generated against an immunogen, typically an immunogen derived from a specific organ, tissue, or cell population, such as, e.g., an organ, tissue, or cell population from an animal at a specific developmental stage.

The use of transparent animals facilitates visualization of developing organs, tissue, and cell populations for antibody selection. Further, the use of small transparent animals (e.g., zebrafish embryos) provide additional advantages, including, for example, maintenance of the animals in individual wells of a microtiter plate (e.g., 96-well plate) and smaller amounts of antibody (e.g., smaller volumes of hybridoma supernatant) for screening. For example, in certain embodiments, hybridoma supernatants can be added directly to wells of a microtiter plate containing the transparent animals (e.g., zebrafish) and screening can be performed as a regular ELISA (antibody incubation, subsequent washing and color development). Therefore, a large number of culture supernatants can be screened simultaneously. A significant advantage of using this feature is that it combines antigen specific screening and in vivo localization in one simplified procedure. Transparent animals that can be used in accordance with the methods provided herein include, for example, teleosts, insect embryos, and nematodes.

Another advantage of the intact transparent animal screening procedure is the capability of simultaneously selecting antibodies against multiple organ-, tissue-, or cell-specific targets. For example, where a specific organ, tissue, or cell population is used as an immunogen to generate antibodies to a particular organ, tissue, or cell-type, mAbs targeting other organs, tissues, or cell populations can be isolated during the screening process.

In one aspect, methods are provided for producing an organ-, tissue-, or cell-specific antibody, the methods generally including the following steps:
(1) immunizing a host animal with an immunogen to generate cells that secrete antibodies;
(2) contacting the antibodies with an intact animal of a species that is transparent through multiple stages of development, or an intact portion thereof (where the intact portion includes a specific organ, tissue, or cell population for which antibody specificity is desired and at least one other organ or tissue), whereby the antibodies permeate the animal or portion thereof; and
(3) detecting the antibodies within the animal to identify an antibody that binds to the specific organ or tissue.
   In certain embodiments of the method, an antigen specifically recognized by the organ-, tissue-, or cell-specific antibody is a developmentally regulated antigen.

Further, in a related aspect, methods are provided for producing an antibody to a developmentally-regulated antigen, the methods generally including the following steps:
(1) immunizing a host animal with an immunogen to generate cells that secrete antibodies;
(2) contacting samples of one of the antibodies with two or more animals of a species that is transparent through multiple stages of development, or an intact portion thereof (where the intact portion includes a two or more tissue types sufficient to allow detection of a predetermined morphogenic change in the multiple developmental stages), whereby the antibody permeates the intact animals or portions thereof;
(3) detecting the amounts and locations of the antibody specifically bound within the animals or portions thereof, and
(4) identifying the antibody as specifically binding to a developmentally regulated antigen based on a difference in the amount and/or location of bound antibody between animals or portions thereof in different developmental stages.

Optionally, the method for producing the antibody further includes generating the immunogen. For example, in certain embodiments in which the immunogen is derived from an organ, tissue, or population of cells from an animal, the method further includes isolating the organ, tissue, or population of cells from the animal (see infra).

A. Antibody Production

1. Immunogen

The immunogen used can be any material, natural occurring or synthetic, that contains antigenic determinants that are at least partially representative of antigenic determinants believed to be present in the transparent animal used for screening the antibodies. Preferably, the immunogen contains antigenic determinants that are at least partially representative of antigenic determinants believed to be present in a specific organ or tissue for which antibody specificity is desired, or antigenic determinants that are present during a specific stage and/or location during development of the transparent animal species. In certain embodiments, the immunogen contains proteins, glycoproteins, proteoglycans, peptides, lipids, glycolipids, and/or carbohydrates. Typically, the immunogen is degradable. In addition, is some embodiments, the immunogen substantially contains molecules that are greater than about 3,000 daltons or greater than about 5,000 daltons, thereby increasing the proportion of molecules that have both a B-cell epitope and an MHC class II-T-cell receptor binding site recognized by a host animal's immune system. In other variations, an immunogen is generated by physically coupling smaller molecules (e.g., less than about 3,000 daltons or less than about 5,000 daltons) to a larger, immunogenic carrier molecule.

In typical embodiments, the immunogen includes cellular or extracellular material from one or more animals. Preferably, the cellular or extracellular material includes antigen determinants from a specific organ, tissue, or population of cells for which antibody specificity is desired. For example, a whole animal (e.g., a whole embryo at a particular developmental stage), or a specific organ, tissue, or population of cells isolated from an animal, can be used to generate the immunogen. Preferably, the cellular or extracellular material is derived from an animal of the species that is transparent through multiple stages of development, i.e., the same species that is used in the method for screen the antibodies against intact animals or intact portions thereof.

In certain preferred variations, cells or tissue are isolated from an organ or tissue region for which antibody specificity is desired. For example, in an exemplary embodiment, the immunogen is derived from cells or tissue isolated from a specific organ or tissue region of a zebrafish (e.g., intestine, pancreas, liver, brain, vascular tissue) at any desired developmental stage, including embryonic and adult stages, and the antibodies generated against the immunogen are subsequently screened using intact zebrafish or intact portions thereof. Optionally, the tissue isolated comprises all or substantially all of an organ for which antibody specificity is desired.

Similarly, in yet other preferred embodiments, cellular or extracellular material for the immunogen is derived from an organ, tissue, or population of cells isolated from an animal at a particular developmental stage for which antibody specificity is desired. For example, the organ, tissue, or population of cells can be isolated from an animal at a developmental stage during which one or more specific morphogenic changes occur such as, for example, angiogenesis (including migration and proliferation of vascular endothelial cells and vessel formation); neurogenesis or axonal pathfinding during peripheral or nervous system development; or other morphogenic events relating to a specific organ including, e.g., spleen, heart, kidney, lung, pancreas, pharynx, esophagus, intestine, gall bladder, lymph nodes, vessels of the circulatory system, or eyes. In addition, in certain embodiments, cells or tissue are isolated from a particular organ or tissue region of the animal (e.g., a specific developing organ or tissue region) for which antibody specificity is desired. The tissue isolated optionally comprises all or substantially all of an organ for which antibody specificity is desired.

In further variations, the isolated organ, tissue, or population of cells is from an animal that is diseased or has been treated with an agent that induces a physiological response (e.g., a drug). Organs, tissues, or cells from diseased animals or animals treated with a physiological response-inducing agent typically contain antigenic determinants that are up- or down-regulated according to the disease state or response to the agent. Thus, an immunogen derived from diseased or agent-treated organ, tissue, or population of cells can be used to produce antibodies that are associated with a particular pathological state or physiological response, typically by screening the antibodies generated against transparent animals that are similarly diseased or agent-treated (see infra). For example, in one specific embodiment, an animal (e.g., a zebrafish) is treated with a drug (e.g., simvastatin) to induce a metabolic response in the liver such as, for example, a pathological induction of fatty liver cells. The liver tissue from the drug-treated animal is then isolated and used to generate an immunogen. As further described infra, the antibodies generated against the immunogen can then screened against drug-treated and non-drug-treated transparent animals to identify an organ-, tissue-, or cell-specific antibody that shows increased or decreased staining of the liver in drug-treated animals relative to non-drug-treated animals.

Where a specific organ, tissue, or population of cells is isolated for preparing an immunogen, any of various microdissection procedures for isolating specific organ(s), tissue(s), or population(s) of cells from a heterogeneous background can be used. Microdissection procedures are generally known in the art. In typical embodiments, the organ, tissue, or population of cells is obtained by microdissection under direct visual inspection (e.g., under a light microscope). In an exemplary embodiment, the microdissection procedure comprises Laser Capture Microdissection (LCM), a method for isolating cells or tissue from specific microscopic regions of tissue sections using infrared laser beams. (See, e.g., Example 1, infra) LCM methods are generally known in the art (see, e.g., Simone et. al., Transplantation 72:164-8, 1999; Simone et. al., Trends Genet. 14:272-6, 1998) and is commercially available (for example, initially developed at the National Institutes of Health (NIH), certain LCM technology is commercialized through a Collaborative Research and Development Agreement (CRADA) partnership with Arcturus Engineering, Inc. to automate and standardize microdissection).

For example, for isolation of cells or tissue by LCM, the starting tissue is typically fixed (e.g., with formalin or ethanol) and embedded in paraffin or frozen, and stained with any of a variety of standard stains to highlight the cell population of interest. An organic polymeric film (e.g., ethylene vinyl acetate (EVA)), impregnated with a dye that absorbs light in the near-infrared spectrum, is attached to a rigid laser cap (e.g., 6 mm laser cap) (see Simone et.al., 1999). The cap is positioned adjacent to an area of interest on the tissue section, and a pulse of near-infrared laser beam is directed to the laser cap. The cap absorbs energy from the laser, is temporarily heated (e.g., to 90° C.), thereby melting and adhering to the underlying tissue. Varying the spot size of the laser within a narrow range (e.g., about 7.5 mm to about 30 mm) can facilitate specificity of the dissection. Further, the laser cap can be moved around on the tissue (e.g., by means of a joystick) to select multiple areas on the same cap. In certain embodiments, about 3000-5000 cells are isolated from a single slide (see Simone et.al., 1998). Typically, the laser cap absorbs most of the energy from the pulse, thereby minimizing transfer of energy to the tissue and decreasing the possibility of heat-induced damage to cellular or tissue components. In some embodiments, a built-in optical system that allows confirmation of the histology of the target area without transferring the slide. Once cells or tissue of interest have been captured, the cap containing the dissected cells or tissue is placed in a suitable container for further processing of the cells or tissue (e.g., a 0.5 ml Eppendorf tube containing lysis buffer).

Once isolated, the cellular or extracellular material can optionally be further processed to further isolate certain subsets of the molecular components (e.g., proteins, glycoproteins, carbohydrates) contained therein. Once purified to the extent desired, the cellular or extracellular material is typically suspended or diluted in an appropriate physiological carrier for immunization or is coupled to an adjuvant.

2. Generation of Antibodies

Methods for generating antibodies to an immunogen are generally known in the art. (See, e.g., Harlow & Lane, Antibodies: A Laboratory Manual (Cold Spring Harbor Laboratory, Cold Spring Harbor, N.Y., 1988.) For the production of such antibodies, various host animals can be immunized by injection with the immunogen. The host animal is typically a vertebrate (e.g., mammal or avian), preferably a mammal (e.g., rabbit, mouse, rat, sheep, goat, camal, llama, and the like), more preferably a rodent, and most preferably a mouse. Various adjuvants can be used to increase the immunological response, depending on the host species, including but not limited to Freud's adjuvant (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (bacille Calmette-Guerin) and Corynebacterium parvum.

For generation of monoclonal antibodies, any technique which provides for the production of antibody molecules by continuous cell lines in culture can also be used. Such techniques include, for example, the hybridoma technique originally developed by Kohler & Milstein (see, e.g., Nature 256:495-97, 1975), as well as the trioma technique, (see, e.g., Hagiwara & Yuasa, Hum. Antibodies Hybridomas 4:15-19, 1993), the human B-cell hybridoma technique (see, e.g., Kozbor et al., Immunology Today 4:72, 1983), and the EBV-hybridoma technique to produce human monoclonal antibodies (see, e.g., Cole et al., In Monoclonal Antibodies and Cancer Therapy, Alan R. Liss, Inc., pp. 77-96, 1985). Human antibodies can be used and can be obtained by using human hybridomas (see, e.g., Cote et al., Proc. Natl. Acad. Sci. USA 80:2026-30, 1983) or by transforming human B cells with EBV virus in vitro (see, e.g., Cole et al., supra). The monoclonal antibody secreted by the transformed or hybrid cell lines can be of any of the classes or subclasses of immunoglobulins, including IgM, IgD, IgA, IgG₁₋₄, or IgE. IgG monoclonal antibodies, typically used in immunochemical applications (including, e.g., immunohistochemical procedures) are preferred.

Repetitive Immunizations Multiple Sites (RIMMS): In one specific embodiment, antibodies are generated using a RIMMS strategy. (See Kilpatrick et al., Hybridoma 16:381-9, 1997.) A RIMMS strategy uses multiple immunizations at sites proximal to draining lymph nodes, followed by isolation of lymphocytes from the draining lymph nodes for cell fusion. (See id.) RIMMS capitalizes on rapid hypermutation and affinity maturation events occurring in B cell populations localized in secondary lymphatic tissue. This strategy typically reduces the time frame for developing monoclonal antibodies: high affinity mAbs can be rapidly produced (e.g., in as little as one month) using this approach. (See, e.g., Wring et. al., J. Pharm. Biomed. Anal. 19:695-707, 1999.) Because of the efficiency of RIMMS, only a few mice are needed, which reduces the quantities of antigen required (30-100 μg), and eliminates the need to monitor polyclonal antisera during the course of immunization. Antisera are typically collected at the time the mice are sacrificed. MAbs developed using RIMMS are affinity matured and can be readily used for ELISAs, immunoblotting assays, immunoprecipitation, and immunostaining.

3. Identification of Antibodies

Once generated, antibodies are screened for organ, tissue, cell, and/or developmental stage specificity by contacting a sample of each antibody with an intact animal of a species that is transparent through multiple stages of development (“transparent species” or “transparent animal”), or an intact portion of such transparent animal, followed by detection of the antibody in the animal to determine its spatial and/or temporal binding pattern in the transparent species.

In certain variations, transparent animals are diseased or have been treated with a drug prior to contact with the antibodies; for these embodiments, detection of the antibodies in the transparent animal allows identification of an antibody that specifically binds an antigen exhibiting a differential expression pattern in a diseased organ, tissue, or cell, or during a physiological response to the drug.

Methods for immunostaining intact transparent animals (e.g., “whole mount” zebrafish or insect embryos) are generally known in the art. (For example, for procedures relating to immunostaining of intact zebrafish, see generally, e.g., Westerfield, The Zebrafish Book: A Guide for the Laboratory Use of Zebrafish (University of Oregon Press, 1993) (hereinafter “Westerfield”). See also, e.g., Argenton et al., Mech. Dev. 87:217-21, 1999; Yager et al., Biochem. Cell Biol. 75:535-50, 1997; Inoue et al., Dev. Dyn. 199:1-11, 1994.) In typical embodiments, antibodies are independently contacted with a transparent animal or intact portion thereof for immunostaining. Generally, animals or intact portions thereof are fixed using an appropriate fixative (e.g., paraformaldehyde or-Dent's fixative (DMSO:methanol=1:3). Preferably, the animals or intact portions thereof are also treated to permeabilize tissues; various reagents suitable for permeabilizing animal tissues are known in the art and include, for example, saponin and Triton X-100. Animals or intact portions thereof can also be treated to quench autofluorescence (e.g., with 1 M NH₄Cl). Animals or intact portions thereof are then rinsed, if necessary, in an appropriate buffer (e.g., a phosphate buffered saline (PBS) solution such as, for example, PBS with Triton X-100 (PBST)), blocked, and stained with an antibody generated as described above (also referred to herein as the “primary antibody”). Typically, the animals or intact portions thereof are rinsed in an appropriate buffer to remove unbound antibody in one or more washing steps. For detection of antigen-bound antibody, animals or intact porioins thereof are preferably contacted with a labeled secondary reagent, typically followed by one or more washing steps to remove any unbound secondary reagent. The labeled secondary reagent can be, for example, a labeled antibody specific for the F_{c} region of the antibodies being screened. The secondary reagent can be either directly labeled (e.g., with a fluorophore or enzyme) or indirectly labeled (e.g., with a hapten or biotin). In alternative embodiments, the primary antibody is directly labeled. Once an animal is stained with an antibody and, if necessary, any suitable secondary reagent(s), the label is detected using any approach suitable according to the particular label used. For example, fluorophore labels can be detected using any suitable means known in the art for detecting the emission wavelength of the particular fluorophore used while also allowing observation of one or more structural features of interest in the transparent animal (e.g., a particular organ or tissue region of interest). Typical methods for detecting fluorescent signals include, for example, fluorescent microscopy (e.g., confocal microscopy).

In one exemplary embodiment comprising the use of zebrafish, zebrafish embryos are fixed in Dent's fixative (e.g., at room temperature for about 3 hours) and dehydrated to 100% ethanol following standard dehydration procedures. Embryos are then permeabilized and rehydrated, and autofluorescence is quenched (e.g., with 1 M NH₄Cl). Embryos are blocked and then incubated with the antibody. After washing with PBST, embryos are incubated with a fluorophore-labeled secondary antibody (e.g., rhodamine-conjugated secondary antibody at, for example, room temperature for 2 hours). Embryos are again washed and then examined by fluorescence microscopy.

In some variations, antibodies are screened against transparent animals in one or more multi-well plates. For example, in one exemplary embodiment, antibodies are screened against zebrafish embryos using the following procedure. Embryos are treated as described above up to the blocking step, and then distributed into a multi-well plate (e.g., 96-well MultiScreen filtration plate (Millipore), 4 embryos per well). Embryos are then incubated with antibody (e.g., supernatant from the hybridoma clones at, for example, room temperature for 4 hours); control embryos incubated with blocking buffer without the antibody are typically set up in the same plate. Unbound antibody is removed using, e.g., a vacuum manifold, and embryos are washed (e.g., with PBST). Fluorophore-conjugated secondary antibody (e.g., rhodamine-conjugated secondary antibody) is then used to stain the embryos, and unbound antibody is again removed. Embryos in each well are examined directly using epifluorescence microscopy. Images are optionally captured using, e.g., a CCD camera. For rapid screening, this process can be semi-automated. For example, an automated liquid dispensing workstation (e.g., MultiProbe II (Packard)) can be used to dispense reagents and solutions. Also, multiscreen filtration plates and a vacuum manifold can be used to process samples.

B. Identification of Target Antigens

In another aspect of the present invention, a target antigen of an antibody generated as set forth above is identified, thereby identifying a protein for drug target validation. Identification of a protein for drug target validation, in addition to the steps described above with respect to the production of organ-, tissue-, or cell-specific antibodies or antibodies to developmentally regulated antigens (see Section II(A)(1-3), supra), further include (1) isolating an antigen specifically bound by the antibody; and (2) using the isolated antigen to identify the protein for drug target validation.

Methods for isolating an antigen specifically recognized by an antibody are generally known in the art. For example, the antigen can be isolated using the antibody in any of various immunopurification procedures. (See generally, e.g., Harlow & Lane (1988), supra; Harlow & Lane, Using Antibodies, A Laboratory Manual (Cold Spring Harbor Laboratory, Cold Spring Harbor, N.Y., 1999).) Generally, immunoaffinity purification methods include coupling (typically, e.g., covalent coupling) of the antibody to a solid matrix (e.g., protein A or protein G sepharose beads), binding of the antigen to the antibody-bead matrix, and elution of the antigen from the antibody-bead matrix. If desired, immunopurification of the antigen can be followed by any additional preparative or purification procedures. In certain variations, gel electrophoresis (e.g., SDS-PAGE) is used as a final purification step.

In another typical embodiment, the antigen is isolated by immunoprecipitation of antigen-antibody complexes. Methods for immunoprecipitation are well-known in the art and generally include preparing a lysate that contains the antigen, contacting the lysate with the antibody to form antigen-antibody complexes, and precipitating the complexes from solution, typically by non-covalently coupling the antibody to a solid matrix (e.g., to protein A or protein G sepharose beads). (See, e.g., Harlow & Lane (1999); Harlow & Lane (1988).) Immunoprecipitation is preferably followed by additional purification procedures to separate the antigen from the antibody (typically, for example, by gel electrophoresis (e.g., SDS-PAGE).

Once isolated, the antigen is used to determine one or more biochemical characteristics of the antigen. In certain embodiments, one or more biochemical characteristics are determined to identify the protein for drug target validation. Various different methods for the biochemical characterization of proteins from biological samples, including methods for identification of proteins, are known in the art and can be used in accordance with the methods as described herein. For example, in one embodiment, an amino acid sequence is determined for the antigen to identify the protein. In certain embodiments, using known procedures, the isolated antigen is cleaved into two or more fragments and an amino acid sequence for one or more fragments is determined. Typically, an N-terminal amino acid sequence of the antigen or a fragment thereof is determined using, e.g., Edman degradation, a method well-known in the art. Edman degradation can be performed, for example, using an automated liquid-phase or gas-phase sequenator. Preferably, a gas-phase sequenator, which can typically analyze picomole quantities of peptide or protein, is used, thereby allowing analysis of, e.g., a single band from a gel.

In other embodiments, the isolated antigen is characterized using mass spectrometry. One suitable mass spectrometry method for analyzing an isolated antigen is the use of Matrix-Assisted Laser Desorption Ionization Time-of-Flight Mass Spectrometry (MALDI-TOF MS) to analyze peptide “fingerprints” produced by fragmenting the proteins with a cleaving agent such as, e.g., an enzyme. Methods for analyzing proteins by MALDI-TOF MS are generally known in the art. (See generally, e.g., Roepstorff, EXS. 88:81-97, 2000; Larsen & Roepstorff, Fresnius J. Anal. Chem. 366:677-90, 2000; Kussmann & Roepstorff, Methods Mol. Biol. 146:405-24:2000; Roepstorff, Pharm. Biotechnol. 145-77, 1995.). For example, in specific embodiments, the isolated protein antigen is isolated by gel electrophoresis (e.g., one- or two-dimensional SDS-PAGE) and fragmented with a cleaving agent such as, for example, an enzyme (for example, digestion can be carried out in a plug of an excised gel and the resulting peptides recovered by elution; by electroelution of the protein from an excised gel plug followed by digestion in solution; by electroblotting the protein onto a membrane (e.g., PVDF) followed by digestion on the membrane; by electroblotting the protein onto a membrane using an immobilized enzyme interposed as the “filling” in an electroblotting “sandwich,” and the like) (see, e.g., id.; U.S. Pat. No. 6,632,339). Once a protein antigen is digested, the peptides are analyzed by MALDI-TOF MS using, e.g., standard procedures. (See, e.g., Roepstorff (2000).) This generally involves contacting the sample with an agent (typically an organic matrix-forming reagent) that absorbs the incident light strongly at the particular wavelength employed. The sample is excited by UV or IR laser light into the vapor phase in the MALDI mass spectrometer. Ions are generated by the vaporization and from an ion plume. The ions are accelerated in an electric field and separated according to their time of travel along a given distance, yielding a mass/charge (m/z) reading. MALDI spectrometers are commercially available and are described in the literature. (See, e.g., Roepstorff (2000).)

Once a MALDI-MS spectrum is generated, the spectral information can, for example, by collected in digital form and the data downloaded to a suitable database research program (e.g., the ExPASy database research program (PeptIdent) for identification of proteins in the Swiss Protein Data Base from mass spectral fingerprints)). The database server can be used to generate the data in a form handleable by computers, thereby providing automated output of MALDI-TOF results. Once the MALDI-TOF spectrum for a protein antigen is obtained, the information can be compared to MALDI-TOF spectra of other proteins in a database to identify a protein having a matching peptide fingerprint, thereby identifying the protein antigen.

In addition to N-terminal sequencing or MALDI-TOF MS, other methods can be used to analyze a protein antigen. For example, fragments obtained following the use of cleaving agent can be analyzed by, e.g., other forms of time-of-flight mass spectrometry, including, for example, liquid chromatography triple quadruple MS, Q-TOF ion trap, or MS-MS.

Optionally, the method for identifying a protein for drug target validation further includes identifying the gene encoding the protein antigen. For example, once an amino acid sequence is determined, or once a protein having an mass spectrometric (e.g., MALDI-TOF MS) fingerprint matching the isolated antigen is identified from a database, a gene can be identified in a genomics database (e.g., GenBank) by searching for a nucleotide sequence that encodes the amino acid sequence or identified protein. The identified nucleic acid sequence can be used, for example, to generate oligonucleotide primers for amplification of the corresponding nucleic acid from, e.g., a genomic or cDNA library. Alternatively, the identified nucleic acid sequence can be used to generate oligonucleotide probes for subsequent hybridization procedures. In another typical embodiment, degenerate oligonucleotide primers, generated based on an amino acid sequence determined for the antigen (such as, e.g., where a partial N-terminal sequence is determined by Edman degradation), are used to generate a cDNA by PCR (e.g., RACE RT-PCR). The nucleotide sequence of the resulting cDNA can then be determined using, e.g., standard nucleic acid sequencing procedures.

Nucleic acids generated as described above can further be utilized as a probe to isolate a complete cDNA or genomic clone. This, in turn, can permit the determination of the gene's complete nucleotide sequence, the analysis of its expression, and the production of its polypeptide product for functional analysis.

Generally, methods relating to molecular genetics and nucleic acid chemistry, including recombinant nucleic acid methods, are well known. Standard techniques such as, for example, those described in Sambrook & Russell, Molecular Cloning: A Laboratory Manual (Cold Spring Harbor Laboratory Press, Cold Spring Harbor, N.Y., 3rd ed. 2001); Sambrook et al., Molecular Cloning. A Laboratory Manual (Cold Spring Harbor Laboratory Press, Cold Spring Harbor, N.Y., 2nd ed. 1989); Short Protocols in Molecular Biology (Ausubel et al. eds., Wiley & Sons, 4th ed. 1999); and Current Protocols in Molecular Biology, Vols. 1-3 (Virginia Benson Chanda ed., John Wiley & Sons, 1994-1998), can be used, e.g., for recombinant nucleic acid methods, nucleic acid synthesis, cell culture, transgene incorporation, and production of a polypeptide product (e.g., for functional analysis).

In yet other embodiments, the method for identifying a protein further includes identifying a species variant of the protein or of the gene encoding the protein. For example, once some or all of the amino acid sequence for the protein antigen has been determined, or once some or all of the nucleotide sequence of the gene encoding the protein is determined, the sequence can be compared with other amino acid or nucleotide sequences in one or more protein or nucleic acid databases using, for example, a sequence comparison algorithm such as, e.g., BLAST or PILEUP (see description of sequence comparison algorithms, Section I, supra). In other embodiments, a nucleic acid probe (e.g., a cDNA probe), having a nucleotide sequence complementary to some or all of the gene encoding the protein, is used to isolate a related nucleic acid from another species. In certain embodiments, the species variant identified is a mammalian (e.g., mouse, rat, or human) variant of the protein or gene. The species variant identified can also be a target for drug target validation.

In another variation of the present invention, the identified protein and/or gene sequences are run on a proteomics or genomics bioinformatics database to identify a functional domain of the identified protein, typically, e.g., by the identification of a signature sequence that is associated with a particular function in other characterized proteins. For example, conserved amino acid motifs and domains associated with particular functions as well as other polypeptides having such motifs and domains can be identified in the National Center for Biotechnology Information Conserved Domains Database, publicly available through the National Center for Biotechnology Information (NCBI) website.

Once a protein for drug target validation is identified, such as, e.g., a species variant of an antigen recognized by an organ-, tissue-, or cell-specific antibody or a species variant of a developmentally regulated antigen, antibodies that specifically bind to the protein can be generated by known methods using the protein or fragments thereof as an immunogen. Such antibodies include, for example, polyclonal antibodies, monoclonal antibodies, chimeric antibodies, single chain antibodies, heavy chain antibody fragments (e.g., F(ab′), F(ab′)₂, Fᵥ, or hypervariable regions), and an F_{ab} expression library (see infra).

C. Antibody Fragments, Chimeric or Humanized Antibodies, Single Chain Antibodies, and Heavy Chain Antibodies

In certain embodiments of the present invention, an antibody fragment (e.g., F(ab′), F(ab′)₂, or Fᵥ fragment), a chimeric or humanized antibody, a single chain antibody, or a heavy chain antibody is produced. In specific embodiments, for example, a chimeric or humanized antibody or antibody fragment is derived from an antibody generated against a protein identified for drug target validation. In a preferred embodiment, a chimeric or humanized antibody or antibody fragment is derived from an antibody generated against a human variant of a target antigen recognized by an organ-, tissue-, or cell-specific antibody produced using a method described herein, or is derived from an antibody generated against a human variant of a developmentally regulated antigen recognized by antibody produced using the methods described herein.

Chimeric antibodies are typically prepared by splicing the non-human genes for an antibody having a desired specificity together with genes from a human antibody molecule of appropriate biological activity. It can be desirable to transfer the antigen binding regions (e.g., F(ab′)₂, F(ab′), Fᵥ, or hypervariable regions) of non-human antibodies into the framework of a human antibody by recombinant DNA techniques to produce a substantially human molecule. Methods for producing such “chimeric” molecules are generally well known and described in, for example, U.S. Pat. Nos. 4,816,567, 4,816,397, 5,693,762, and 5,712,120; International Patent Publications WO 87/02671 and WO 90/00616; and European Patent Publication EP 239 400). Alternatively, a human monoclonal antibody or portions thereof can be identified by first screening a human B-cell cDNA library for DNA molecules that encode antibodies that specifically bind to an identified target antigen or species variant thereof (e.g., preferably a human variant of a protein for drug target validation) according to the method generally set forth by Huse et al. (Science 246:1275-81, 1989). The DNA molecule can then be cloned and amplified to obtain sequences that encode the antibody (or binding domain) of the desired specificity. Phage display technology offers another technique for selecting antibodies that bind to identified target antigens, fragments, derivatives, or analogs thereof. (See, e.g., International Patent Publications WO 91/17271 and WO 92/01047; Huse et al., supra). Methods for making humanized antibodies are also disclosed in Queen et al., Proc. Natl. Acad. Sci. USA 86:10029-33, 1989; and WO 90/07861, U.S. Pat. Nos. 5,693,762, 5,693,761, 5,585,089, 5,530,101, and 5,225,539.

Techniques described for the production of single chain antibodies (see, e.g., U.S. Pat. Nos. 4,946,778 and 5,969,108) can be adapted to produce single chain antibodies specific for an organ-, tissue-, or cell-specific antigen or species variant thereof. For example, an additional aspect of the invention utilizes the techniques described for the construction of a F_{ab} expression library (see, e.g., Huse et al., supra) to allow rapid and easy identification of monoclonal F_{ab} fragments with the desired specificity for a protein identified for drug target validation, or fragments, derivatives, or analogs thereof.

The antibodies also can be heavy chain antibodies. Immunoglobulins from animals such as camels, dromedaries, and llamas (Tylopoda) can form heavy chain antibodies, which comprise heavy chains without light chains. (See, e.g., Desmyter et al., J. Biol. Chem. 276:26285-90, 2001; Muyldermans & Lauwereys, J. Mol. Recognit. 12:131-40, 1999; Arbabi Ghahroudi et al., FEBS Lett. 414:521-26, 1997; Muyldermans et al., Protein Eng. 7:1129-35, 1994; Hamers-Casterman et al., Nature 363:446-48, 1993.) The variable region of heavy chain antibodies are typically referred to as “VHH” regions. (See, e.g., Muyldermans et al., TIBS 26:230-35, 2001.) The VHH of heavy chain antibodies typically have enlarged or altered CDR regions, as such enlarged CDR1 and/or CDR3 regions. Methods of producing heavy chain antibodies are also known in the art. (See, e.g., Arbabi Ghahroudi et al., supra; Muyldermans & Lauwereys, supra.)

Antibody fragments that contain the antigen-binding site can be generated by known techniques. Such fragments include, for example, the F(ab′)₂ fragment which can be produced by pepsin digestion of the antibody molecule, the F(ab′) fragments which can be generated by reducing the disulfide bridges of the F(ab′)₂ fragment, the F_{ab} fragments which can be generated by treating the antibody molecule with papain and a reducing agent, and Fᵥ fragments. Recombinant Fᵥ fragments can also be produced in eukaryotic cells using, for example, the methods described in U.S. Pat. No. 5,965,405.

D. Blood Vessel-specific Monoclonal Antibodies and Related Compositions

In specific variations of the present invention, monoclonal antibodies that specifically recognize zebrafish blood vessels are provided. Generally, the blood vessel-specific monoclonal antibodies are produced according to the methods described above for generating organ- or tissue-specific antibodies (see Section II(A), Antibody Production, supra) using blood vessel tissue as an immunogen.

For example, in one exemplary embodiment, the blood vessel-specific monoclonal antibodies are produced according to the following procedure. To generate an immunogen, blood vessels are extracted from zebrafish. Typically, zebrafish used are embryos (e.g., 3 month old embryos generated, for example, by to the natural pair-wise mating according to Westerfield, supra). Blood vessels are typically identifiable by their natural reddish black color using microscopy and can be exposed, for example, by dissecting zebrafish longitudinally from the abdominal side of the vertebra. Typically, the blood vessels are separated from the vertebra. Alternatively, blood vessel tissue can, for example, be isolated by LCM (see generally, e.g., Section II(A)(1), Immunogen, supra; Example 1, infra). Following dissection, blood vessel tissue is typically transferred to an appropriate buffer for further preparative procedures. For example, blood vessel tissue can be stored in PBS, homogenized, centrifuged (e.g., at ˜1,000 rpm for 2 minutes), and the supernatant recovered for use as immunogen. Blood vessels from multiple embryos (e.g., ˜50) are typically pooled to prepare the immunogen. The immunogen is used to immunize mice using, for example, standard immunization procedures or RIMMS (see Section II(A)(2), Generation of Antibodies, supra; Example 1, infra). Pre- and post-immunization sera are typically collected and screened against zebrafish using the whole mount immunostaining technique described above (see Section II(A)(3), Identification of Antibodies, supra) to identify mice with positive serum. Mice with positive serum are used to produce hybridomas (see generally, e.g., description of hybridoma, Section II(A)(2), supra; Harlow & Lane (1988)). Supernatant of the clones are screened against intact zebrafish embryos by immunostaining using, e.g., a semi-automated procedure to screen against embryos in multi-well (e.g., 96-well) plates (see Section II(A)(3), Identification of Antibodies, supra). Supernatants that demonstrate specific staining of blood vessels relative to non-vessel tissues. Once identified as positive, hybridoma wells are further diluted and plated to obtain single-cell clones.

Typical zebrafish blood vessel-specific monoclonal antibodies are those specifically recognizing the dorsal aorta, heart, head region vessels, intersegmental vessels, posterior cardinal vein, and/or subintestinal vessels. In some variations, the monoclonal antibody specifically recognizes angiogenic vessels, such as, for example, the intersegmental vessels and head region vessels, but does not specifically recognize major vasculogenic vessels, including, e.g., the dorsal aorta and posterior cardinal vessel. Exemplary antibodies include Phy-V001, Phy-V002, Phy-V003, Phy-V004, Phy-V005, and Phy-V006.

The foregoing antibodies as described in Section II(A-D) can be used in methods known in the art relating to the localization and activity of the target antigen, including a protein identified for drug target validation (e.g., for imaging proteins, measuring levels thereof in appropriate physiological samples, in diagnostic methods, modulating a biological activity, and the like).

Accordingly, in another aspect of the present invention, a kit for detection of the presence of target antigen is provided. For example, in certain embodiments, the kit comprises the antibody and at least one of the following reagents: a blocking reagent (e.g., a protein solution such as, for example, bovine serum albumin), a washing reagent (e.g., phosphate- or Tris- buffered saline optionally comprising a surfactant such as, for example, Tween-20), and a reagent capable of detecting the presence of the antibody (e.g., a labeled secondary antibody such as, for example, a fluorescently-labeled antibody recognizing mouse IgG heavy chain).

III. Methods of Identifying Agents that Modulate Angiogenesis

In another aspect of the present invention, methods are provided for identifying agents that modulate angiogenesis. Using the methods, an agent is screened for activity in modulating angiogenesis either independently or in combination with one or more other agents. Further, in addition to screening a single agent, the methods can be used to screen multiple agents such as, for example, agents from a compound library. Compound libraries can include, for example, a historical collection of compounds synthesized in the course of pharmaceutical research; libraries of compound derivatives prepared by rational design (see generally, Cho et al., Pac. Symp. Biocompat. 305-16, 1998; Sun et al., J. Comput. Aided Mol. Des. 12:597-604, 1998), such as, e.g., by combinatorial chemistry; natural products libraries; peptide libraries, and the like. Methods of making combinatorial libraries are generally known in the art and are described in, for example, U.S. Pat. Nos. 5,958,792; 5,807,683; 6,004,617; and 6,077,954.

In one embodiment, the screening methods generally include the following steps:
(1) contacting the agent with a teleost;
(2) contacting the teleost with an antibody that specifically recognizes vascular endothelial cells in teleosts;
(3) contacting a control teleost with the antibody, or antigen-binding fragment thereof, that specifically recognizes vascular endothelial cells in teleosts, where the control teleost has not been contacted with the agent;
(4) detecting the antibody or fragment thereof in the teleost contacted with the agent and the control teleost to visualize an angiogenic process; and
(5) comparing the angiogenic process in the teleost contacted with the agent with that in the control teleost to determine whether the agent modulates angiogenesis.

In certain variations of the screening method, the teleost is of a species that is transparent through multiple stages of development. The use of a transparent species facilitates the observation of the angiogenic process in intact animals or intact portions thereof in certain embodiments of the methods described herein. In one exemplary embodiment, the teleost is a zebrafish.

The teleost can, for example, be of a predetermined developmental stage during which one or more angiogenic processes are typically observed (for example, in the zebrafish, the major angiogenic vessels typically begin forming at about 1.2-1.5 days post fertilization (dpf), and tissue angiogenic vessels are typically observed at about 4 dpf when the intestines and liver form). In another embodiment, alternatively or in additionally, the teleost is maintained in the presence of conditions (e.g., a specific agent) that either induces or inhibits one or more angiogenic processes. The agent being screened for modulatory activity (test agent) can be added, e.g., after treatment with, at a particular time point during treatment with, contemporaneously with addition of, or even prior to addition of the angiogenesis-inducing agent.

Once the teleost is contacted with the agent being screened for modulatory activity (test agent), the teleost is maintained viable for a period of time sufficient to allow detection of any modulatory (e.g., angiogenic or anti-angiogenic) effects associated with the presence of the agent relative to a control teleost (i.e., that is not contacted with the test agent). Thus, typically, following contact of the teleost with the test agent, the intact teleost, or an intact portion thereof,is maintained under culture conditions for a time period that is sufficient to allow one or more angiogenic processes to progress to a detectable degree, assuming the absence of an anti-angiogenic agent. For example, in specific embodiments in which the teleost is a zebrafish embryo, following contact of a dechorionated zebrafish embryo with the test agent (e.g., at about 1 dpf), the embryo, including the attached yolk ball, is maintained for, e.g., 1, 2, 3, 4, or 5 days in culture conditions that include, for example, embryo water (prepared, e.g., by dissolving 5 g of Instant Ocean Salt in 25 liters of distilled water) at 28° C.

Following the desired treatment, the teleost is processed for staining with the vascular endothelial cell-specific antibody. In one embodiment, developmental and cellular processes are terminated at a desired time point by, for example, flash-freezing the tissues or treating the animal with a fixative (e.g., paraformaldehyde). Tissues can, for example, be sectioned prior to contact with an antibody. Procedures for tissue sectioning are generally known. For example, tissues can be prepared following known procedures for frozen tissue sectioning or sectioning of paraffin-embedded tissues. (See, e.g., Harlow & Lane (1999).) The samples are then contacted with the antibody using, for example, standard procedures known in the art for immunostaining of tissue sections. (See, e.g., id.) In an alternative embodiment, an intact teleost, or intact portion thereof, is contacted with the antibody. Typically, the intact animal or intact portion thereof is permeabilized any of various suitable permeabilizing agents such as, e.g., saponin or Triton X-100. The teleost is then stained with the antibody following, for example, standard procedures known in the art for whole mount immunostaining. (See, e.g., Examples 4 and 5.)

In preferred embodiments, the antibody is a monoclonal antibody. The antibody can, for example, be an antibody identified as specific for vascular endothelial cells using a method for production of an organ-, tissue-, or cell-specific antibody as described herein. In exemplary embodiments, the antibody is Phy-V001 mAb, Phy-V002 mAb, Phy-V003 mAb, Phy-V004 mAb, Phy-V005 mAb, or Phy-V006 mAb (see infra).

The antibody can be detected by, e.g., any of various methods-known in the art. In certain embodiments, for example, the antibody or fragment thereof is modified to include a detectable label. In an exemplary embodiments, the label is a direct label such as, for example, a fluorophore (e.g., fluorescein, rhodamine, or phthalocyanine dyes). In other embodiments, an indirect label is used (e.g., a hapten or biotin). In yet other embodiments, an antibody that is not modified to include a label is detected using a secondary reagent such as, for example, a labeled secondary antibody (e.g., an anti-IgG antibody). Once a teleost is stained with an antibody and, if necessary, any suitable secondary reagents, the label is detected using any approach suitable according to the particular label used. For example, fluorophore labels can be detected using any suitable means known in the art for detecting the emission wavelength of the particular fluorophore used while also allowing observation of one or more structural features of interest in the teleost (e.g., a particular organ or tissue region of interest). Typical methods for detecting fluorescent signals include, for example, fluorescent microscopy (e.g., confocal microscopy).

The pattern and level of antibody staining in the teleost contacted with test agent and the control teleost are examined to determine the relative progression of one or more angiogenic processes, thereby determining whether the agent is a modulator (e.g., stimulator on inhibitor) of angiogenesis. For example, an increased level of an angiogenic process in the teleost contacted with the test agent as compared with the control teleost would identify an agent as a stimulator of angiogenesis, and vice versa.

For example, in a specific embodiment, an agent is screened for activity in modulating angiogenesis according to the following procedure. Zebrafish embryos at a predetermined stage (e.g., about 1 dpf, about 2 dpf, or about 3 dpf) are harvested and incubated with one or more predetermined concentration(s) of the agent continuously for a predetermined time period (e.g., about 12, about 24, about 48, or about 72 hours). Effects of the agent are then examined, preferably at 2 dpf or 3 dpf, by fixing the embryos at the desired time point and then immunostaining with Phy-V001 or Phy-V002 mAb. Zebrafish embryos are fixed in Dent's fixative (e.g., at room temperature for about 3 hours) and dehydrated to 100% ethanol following standard dehydration procedures. Embryos are then permeabilized and rehydrated, and autofluorescence is quenched (e.g., with 1 M NH₄Cl). Embryos are blocked and then incubated with Phy-V001 or Phy-V002 mAb. After washing with PBST, embryos are incubated with a fluorophore-labeled secondary antibody (e.g., rhodamine-conjugated secondary antibody at, for example, room temperature for 2 hours). Embryos are again washed and then examined by fluorescence microscopy. In certain embodiments, the immunostaining procedure is carried out in multi-well plates and is optionally semi-automated (see, e.g., description of multi-well immunostaining procedure, Section II(A)(3), supra; Examples 5 and 6, infra).

Optionally, the method for screening an agent for activity in modulating angiogenesis further includes contacting a second animal with the agent and monitoring the second animal for a physiological response to the agent that is indicative of toxicity. Physiological responses that are indicative of toxicity can include, for example, non-specific inhibition of non-angiogenic processes (including, e.g., a general inhibition of embryo growth), hemorrhage, edema, or neuropathy. For example, to assess toxicity of an agent in, e.g., a zebrafish embryo, the embryo can be treated with the agent at a predetermined embryonic stage (e.g., 1 dpf) at at least one concentration of the agent, typically at multiple concentrations, for a period of time (e.g., about 1, typically about 2, more typically about 3, and preferably at least 4 days). A physiological change indicative of toxicity is them measured. For example, in specific embodiments, growth inhibition is assessed by measuring, e.g., body length; hemorrhage is assessed by measuring, e.g., blood pooling outside the circulation circuitry; edema is assessed by measuring expansion of organ structure (e.g., heart); or neuropathy is assessed by measuring necrosis in the head region.

A further understanding of the present invention will be obtained by reference to the following description that sets forth illustrative embodiments.

### EXAMPLE 1

Generation of Organ-specific Antibodies to Zebrafish Pronephros and Pancreas

Zebrafish Pronephros: As a model system, zebrafish offer distinct experimental advantages for studying pronephric development. The striking similarity of pronephric cell types to those found in higher vertebrates, as well as the conservation of pronephros-specific gene expression patterns, suggest that insights gained from studies in zebrafish will be broadly applicable to studies on cell differentiation in human kidney. Studies of renal development may contribute to novel therapeutic approaches for renal disease and advance treatments for long-term degenerative kidney disease. Several immunosuppressive drugs, including cyclosporin A, are known to cause severe nephrotoxic side effects (see de Mattos et.al., 2000; Bennett W M, 1998). Although it was recently reported that Flt-1 and KDR/Flk-1 are upregulated in cyclosporin induced nephrotoxicity, the key molecules and signaling pathways involved in the nephrotoxic side effects of many drugs are still not clearly distinguished

Although the pronephros develops as early as two days post-fertilization, two weeks is the earliest suitable time point to microdissect the organ, because at this stage it is easy to distinguish the renal tubules of the pronephros from the surrounding hematopoeitic tissue and the relevant filterative tissue is abundant (see Willett et. al., Dev. Dyn. 214:323-36, 1999). Extracts from samples dissected from two to sixteen week zebrafish are pooled for use as immunogens. This is an effective approach for generating mAbs targeting adult as well as maturing pronephros and also to identify tissue-specific, developmentally regulated antigens.

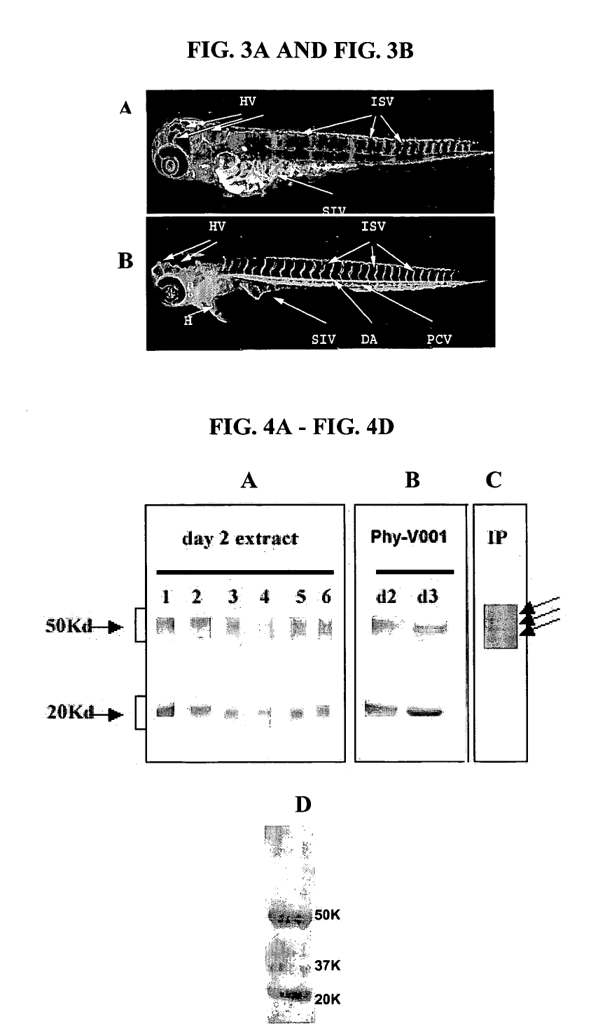

Zebrafish Pancreas: The recent emergence of the zebrafish as a highly suitable model organism for vertebrate development has prompted investigation of its potential for studying pancreatic organogenesis (Biemar et.al., Dev. Biol. 230:189-203, 2001). Pancreas of the zebrafish embryo is located on the right side of the embryo and is composed of a single islet embedded in exocrine tissue (FIG. 3). The adult zebrafish pancreas is a less coherent structure than the mammalian pancreas because it has several prominent islets that are located near the proximal end of the pancreatic duct and accessory islets and exocrine tissue are scattered along the intestinal mesentery in close proximity to the pancreatic ducts (Pack et.al., Development 123:321-8, 1996). These areas are easily located on day five zebrafish sections and microdissection therefore, will be straightforward. Microdissected samples from one, two, four, eight and sixteen week old zebrafish pancreas are pooled for use as immunogens to produce specific mAbs targeting adult and maturing pancreas.

Generating mAbs targeting zebrafish pronephros and pancreas will contribute to understanding the cellular patho-physiology of these organs and assist in establishing drug toxicity screening assays.

Using LCM (Laser Capture Microdissection) technology, tissues from the pronephros of two, four, eight and sixteen week zebrafish are microdissected and extracts are pooled for use as immunogens. RIMMS (Repetitive Immunizations Multiple Sites) is then used to generate mAbs targeting both adult organs and the maturing pronephros. For pancreas, sections from seven day old zebrafish embryos are used. These samples are pooled with samples microdissected from two, four, eight and sixteen week zebrafish pancreas to generate mAbs targeting adult as well as developing pancreas using the RIMMS method.

For RIMMS, six mice are used for each immunogen. Following immunizations, the lymph nodes are dissected out for use in fusions to generate hybridomas. The supernatants containing the mAbs secreted by hybridomas are screened using a whole zebrafish assay format. Tissue and stage specific hybridomas for both embryos and adult zebrafish are sub-cloned for further characterization and purification.

1. Use of LCM Technology to Microdissect Zebrafish Organs to Generate Tissue-Specific Immunogens

Zebrafish Tissue Preparation: Zebrafish (male and female) aged two, four, eight, and sixteen weeks post-fertilization are euthanized following anesthetization using Tricane methanesulfonate. Zebrafish are fixed in 4% paraformaldehyde/5% sucrose/Phosphate-buffered saline pH 7.4 (PBS) for 3 hours at room temperature, washed in 5% sucrose/PBS and cryoprotected in 30% sucrose/PBS overnight at 4° C. Fish are subsequently permeated in 1:1 OCT/30% sucrose/PBS overnight and 100% OCT overnight at 4° C. and embedded in OCT compound (Electron Microscopy Sciences, Fort Washington, Pa.) to generate blocks for cryosectioning and subsequent LCM.

Cryosectioning: Typically, 10-12 μm serial sections will are in a standard cryostat and three sections are each mounted on Superfrost Plus (Fisher Scientific, Tustin, Calif.). The slides are kept in the cryostat or on dry ice if LCM is to be performed the same day. Alternatively, they are stored at −80° C. until used. Staining is performed as close as possible to the scheduled LCM transfer time using solution baths that are replaced regularly. Staining is performed as follows: (1) 70% ethanol wash for 20 seconds, (2) wash briefly in distilled water, (3) Mayer's hematoxylin (1-2 min), (4) wash in distilled water to rinse excess stain, followed by (5) 70% ethanol wash for 20 seconds and immediately used for LCM. The timing of ethanol treatment and staining can be modified as necessary to ensure optimal capture of relevant tissues. Further, 6 to 8 μm sections can also be used to enhance visualization of the tissues; in this case, the number of slides required to isolate similar amounts of protein will increase.

Microdissection: LCM is performed using a Arcturus Pix-cell II. Briefly, the zebrafish pronephros and the pancreas are located under the microscope and dissected out onto the caps and transferred into Eppendorf tubes containing 50 μl lysis buffer (50 mM Tris, pH 8.0, 250 mM NaCl and 0.5% NP-40) containing protease inhibitors (1 mM PMSF, 0.05 mM leupeptin, 0.01 mM pepstatin) and incubated for 10 minutes at room temperature.

The number of cells visible within a 30 mm diameter field depends on the type of tissue and ranges from an average of 20 cells within a human lymph node to 1 cell in fibrous tissue. Typically, about 4-7 μg protein /mm² area can be obtained from a 10 μm thick-rat liver section (Shibutani et al., Lab. Invest. 80:199-208, 2000). For different tissues, the number of laser-targeted fields differs. Therefore, the method is typically standardized for the zebrafish tissue section(s) targeted. RIMMS method typically uses about 80 μg of protein per immunization per mouse (typically with eight sites per immunization at about 10 μg per site). Therefore, a 20 mm² area is typically dissected out (at 4 μg protein/mm² area) to obtain protein for use as immunogen.

The amount of protein extracts obtained is estimated using standard techniques and stored at −80° C. until used for immunizations. Typically, LCM technology is used to generate enough protein so that the same batch can be used for RIMMS immunizations. This reduces batch to batch variation in the protein profile obtained.

2. Use of RIMMS to Rapidly Generate Organ Specific mAbs

RIMMS Strategy: RIMMS immunizations and fusion are done according to the procedure of Kilpatrick et al., Hybridoma 16:381-9, 1997.

Generation and Isolation of the P3XBcl-2-13 Fusion Partner: The human Bcl-2 coding sequence is amplified using sequence specific primers by RT-PCR using mRNA from human prostate (BD Biosciences, Palo Alto, Cailf.) and sub-cloned into pCI-neo vector (Promega, Madison, Wis.) using standard protocols. The construct is subsequently transfected into the non-secretory myeloma cell-line P3X63/Ag8.653 grown in established conditions (Kearney ei.al., J. Immunol. 123:1548-50, 1979) using lipofectamine PLUS (Invitrogen, Carlsbad, Cailf.) according to manufacturer's instructions. Bcl-2 expression levels are determined by standard Western blotting procedures (Kilpatrick et.al., supra) using murine mAbs reactive to human Bcl-2. Stable P3XBcl-2-13 lines carrying the human Bcl-2 are used as fusion partners to generate hybridomas.

Repetitive Immunizations: Six Balb/c female mice or SJL male mice (Charles River Laboratories, Wilmington, Mass.) are used for each immunogen. Zebrafish pronephros and pancreas protein lysates prepared subsequent to LCM are estimated using standard procedures and used as immunogens. Typically about 3-10 μg of antigen is diluted in PBS, emulsified with FCA (Sigma, St. Louis, Mo.), and 40-50 μl is injected into each of 8 sub-cutaneous sites proximal to the draining lymph nodes. A total of 5 RIMMS immunizations are done over a period of 7-11 days.

Polyethylene Glycol (PEG)-Induced Somatic Fusion of Immune PLN Cells: The mice are euthanized using isoflurane. A single cell suspension of lymphocytes is then harvested bilaterally from popletial, superficial inguinal, axillary and brachial lymph nodes and fused with P3XBcl-2-13 cells at a ratio of 2.5:1 using modifications of a published protocol (Kilpatrick et.al., Hybridoma 14:355-9,. 1995). Briefly, lymphocytes are teased into RPMI-1640 (Invitrogen). 0.5 to 1 ml of 34% polyethylene glycol 1450 (ATCC, Rockville, Md.) diluted in serum free RPMI-1640 are added to the mixed cell pellet in a 50 ml conical tube over the course of 1 min. in a 37° C. water bath. The PEG-cell suspension is mixed gently for 1 min. in the water bath and diluted by slowly adding 13 ml RPMI-1640, followed by centrifugation for 7 min. at 400 g. Pelleted cells are re-suspended at 2×10⁴ cells per ml in 48-120 ml of hybridoma medium (HM) containing a mixture of 50:50 EXCELL-610 (JRH BIOSCIENCES, Lenexa, Kans.) and RPMI-1640, supplemented with 10% FBS, 10% Origen Cloning Factor (Igen, Rockville, Md.), 2 mM 1-Glutamine, 100 μl/ml penicillin and 0.01 mM 2-mercaptoethanol. One ml/well of the fusion suspension are distributed into 16 mm by 24 well plates. After 24 hours in culture, 1 ml HM supplemented with 2× HAT (Hypoxanthine, Aminopterine and Thymidine (Invitrogen) are added to each well. Media is changed 72 hours later to HM-HAT containing 200 μg/ml G418.

The clones are grown in 24 well plates and screened for stage and organ specificity, as described below. Clones positive for adult and embryonic zebrafish pronephros and pancreas are cloned by limiting dilution according to established procedures (See mAbs: A Practical Approach, (Phil S. Shepherd and Chris Dean, eds., Oxford University Press, Inc., pp. 1-479).

Embryo Collection for Screening for Organ-specific Clones: Embryos are generated by natural pair-wise mating, as described in Westerfield, supra. 4-5 pairs are set up for each mating, and, on average, 100-150 embryos per pair are generated. Embryos are maintained in embryo water (5 g of Instant Ocean Salt in 25 liters of distilled water) at 27° C. Healthy embryos are dechorionated by enzymatic digestion with lmg/ml protease (Sigma, St. Louis, Mo.) for 5 minutes at room temperature. The embryos are then washed 5 times in embryo water. Because the fish embryo receives nourishment from an attached yolk ball, no additional maintenance is required.

Whole Mount MAb Staining: Briefly, embryos (day 1 to day 6 post-fertilization) are fixed by 4% paraformaldehyde (in PBS) containing 0.1% saponin for 2 hours at room temperature, washed by PBS, then dehydrated gradually into 100% EtOH following standard dehydration procedures. The fixed embryos are stored in 100% EtOH at−20° C. for at least 1 hour for permeablization, and then rehydrated gradually into PBS containing 0.1% saponin (PB SS) following standard procedures (Westerfield, 1993). The embryos are blocked in a solution containing 3% BSA/PBS at room temperature for at least 2 hours, and then incubated with the supernatants containing the putative mAbs overnight at 4° C. After washing with PBSS, secondary mAbs (whole Ig) conjugated to horseradish peroxidase (HRP) are incubated with embryos at room temperature for at least 2 hours. After washing once with PBSS, embryos are washed extensively in PBS. Color is developed using the enhanced Di-amino Benzidene (DAB) substrate kit (Pierce, Rockford, Ill.) according to manufacturer's instructions and examined using Nikon E-600 dissecting microscope (Micro Video Instruments, Avon, Mass.)

### EXAMPLE 2

Generation of Antibodies to Antigens Involved in the Zebrafish Digestive System

MAbs are generated to identify antigens involved in the zebrafish digestive system for use, e.g., in drug screening assays or as a research tool for the zebrafish research community. Embryos between 18 hpf and 120 hpf are used as a source for generating immunogens for mAb production: the digestive system begins developing around 18 hpf and comprises a fully functional gut, liver and pancreas by 120 hpf. These embryos are processed to segregate the liver, gut, and accessory organs associated with the digestive system and subsequently homogenized and used as immunogens. Hybridomas are generated using conventional procedures and the supernatant containing the mAbs secreted by hybridomas are screened using a whole zebrafish assay format. MAbs targeting antigens involved in development of the digestive system and its accessory organs are further characterized to determine temporal expression patterns.

Unique and conserved aspects of the digestive system ofzebrafish: During organogenesis, the endoderm forms the epithelial lining of the primitive gut tube from which the alimentary canal and associated organs, such as the liver and pancreas, develop. Despite the physiological importance of these organs, current knowledge of the molecules regulating their development is limited. However, evidence suggests that common molecular determinants regulate liver, pancreas, pharynx, and esophagus development in zebrafish and mammals. Morphogenesis of the digestive tract and accessory digestive organs begins around 18 hpf and by about 58 hpf the common ductal system of the liver and pancreas is contiguous with the digestive tract. Therefore, this time frame is important for establishing molecular signals associated with the formation of the digestive system and related organs. Subsequently, during larval development, there is further growth and differentiation of all digestive organ primordia and by 120 hpf the digestive system is fully functional. Histological analysis and gene expression patterns within the zebrafish endoderm suggest a model of digestive tract morphogenesis and organ progenitor specification. Identifying the molecular targets involved in the organogenesis of the digestive system has implications for research on human diseases, such as diabetes, liver cirrhosis, and cancer.

Zebrafish as a modelfor studying liver disease: Because the liver is the major organ that metabolizes drugs and xenobiotic reagents, it is the most general target for adverse drug effects. A single screening platform for liver response to potential therapeutic compounds is of great interest to pharmaceutical toxicologists. The zebrafish has previously been extensively used to study the toxic effects of environmental pollutants (see Boumann & Sander, J. Exp. Zool. 230:363-376, 1984; Mizell & Romig, J. Dev. Biol. 41:411-23, 1997). Using lethality, embryo survival rate, behavior and microscopic examination of organ malformation as general assay parameters, these studies demonstrated that zebrafish exhibits good dose-responsiveness to toxicity and is a suitable animal model for screening chemicals. Similar to mammals, zebrafish liver is the major organ for drug metabolism and its morphology is sensitive to drug treatment. Moreover, zebrafish exhibit similar phenotype for liver fatty acid disease as humans: treatment of zebrafish with simvastatin, a well characterized liver toxicant, shows that zebrafish exhibit simvastatin metabolic response and pathologically display typical fatty liver characters found in humans.

1. Immunogen-isolation of Zebrafish Liver, Rut, and Related Organs

Embryo Maintenance, Collection, and Processing: Embryos are generated as described in Example 1 (see also Westerfield, supra). Embryos are maintained in embryo water (5 g of Instant Ocean Salt in 25 liters of distilled water) at 27° C. for approximately 18 hours (20 somite stage) before sorting for viability. Since, the digestive system begins developing around 18 hpf and comprises a fully functional gut, liver, and pancreas byl 20 hpf, embryos between ages 18 hpf and 120 hpf are harvested at 24 hour intervals (approximately five 24 hour time points—3000 embryos per time point). These will includel 18 hpf, 42 hpf, 66 hpf, 90 and 120 hpf. The yolk is removed using a conventional gel loading pipet tip (Bio-Rad, California) attached to a Gilson p200 pipet man (VWR Scientific, Massachusetts).

Yolk consists of a number of proteins that are potentially antigenic. Therefore, yolk removal is one procedure for generating immunogen. Following yolk removal, the head and the tail of the embryos are removed using an 18 gauge needle. This leaves behind the region, predominantly consisting of the developing gut, the pancreas, and the liver.

Zebrafish Protein Extracts and Estimation of Total Protein: Total protein is extracted from embryos for immunogen. Embryos from each stage are collected, processed as described above, and put through two freeze thaw cycles, at −80° C. and room temperature, respectively, at 20 minutes for each cycle. Subsequently, the embryos are lysed in a Nonidet P-40 lysis buffer (150 mM NaCl, 1.0% Nonidet P-40 (Sigma, Missouri), 50 mM Tris, pH 8.0, see Harlow and Lane, Antibodies: A Laboratory Manual (Cold Spring Harbor Laboratory Press, 1988)) containing a protease inhibitor cocktail set (Calbiochem, California) and homogenized by sonication (Branson Ultrasonics Corp., Connecticut) at 4° C. to extract proteins. The total protein in the extracts is estimated by Bradford's method (Bradford, 1976) using an assay kit (BIO-RAD, California), according to manufacturer's instructions. The immunogens are stored at −80° C. until use.

2. Generation of Hybridomas and Screening for Organ Specific Targets

Immunizations: The immunogens generated as described above are used to generate mAbs (Cocalico Biologicals, Inc., Pennsylvania). Three mice are used for immunization. The sera from these mice are used to titer the antibody response using a whole mount immunostaining procedure described below. For fusion (Cocalico Biologicals, Pennsylvania), two mice that have the highest titer of antibodies targeting gut and the accessory organs are used. Following fusions, the hybridoma culture supernatants are subjected to primary screening, as described below. Relevant positive clones are sub-cloned (Cocalico Biologicals, Pennsylvania) and further characterized.

Screening Culture Supernatants: For screening assays, embryos are harvested between 18 hpf and 120 hpf at 24 hour intervals (approximately five “24 hour” time points), fixed by 4% paraformaldehyde (in PBS) containing 0.1% saponin for 2 hours at room temperature, washed by PBS, then dehydrated gradually into 100% EtOH following standard dehydration procedures. The fixed embryos are stored in 100% EtOH at −20° C. for at least 1 hour for permeabilization, and then rehydrated gradually into PBS containing 0.1% saponin (PBSS) following standard procedures (Westerfield, supra). Three embryos are dispensed into each well of a 96 well plate for screening. The embryos are blocked in a solution containing 2% BSA/PBS at room temperature for at least 2 hours, and then incubated with undiluted culture supernatants overnight at 4° C. After washing with PBSS, the embryos are incubated with a secondary antibody conjugated to Rhodamine (Sigma, Missouri) for 2 hours at room temperature. After extensive washing with PBSS, the embryos are examined under a Nikon E-600 epifluorescence microscope (Micro Video Instruments, Massachusetts).

Each tissue culture supernatant is screened with embryos of five “24 hour” time points between 18 hpf and 120 hpf: 18 hpf, 42 hpf, 66 hpf, 90 hpf and 120 hpf. Supernatants are screened for tissue specific expression as well as changes in temporal expression patterns. Hybridoma clones showing tissue specific (liver, gut, and pancreas) and/or showing an interesting temporal change(s) in expression pattern is selected for further characterization. This whole animal screening protocol permits simultaneous isolation of multiple marker proteins.

3. Characterize Relevant Target Proteins for Use in Drug Screening Assays and for Use as Reagents

MAbs in culture supernatants that show tissue specific staining and/or those that show temporal changes in staining patterns, indicating that they may target antigens involved in organogenesis, are further characterized. Preliminary characterization includes Western blots on extracts prepared from embryos between 18 hpf and 120 hpf: 18 hpf, 42 hpf, 66 hpf, 90 hpf and 120 hpf (same time points as was used for our whole mount screening described above). This confirms the expression patterns seen on whole mount immunostaining and is also useful in identifying the molecular size of the target protein(s).

Zebrafish Protein Extracts and Estimation of Total Protein: Embryos between ages 18 hpf and 120 hpf are harvested at 24 hour intervals (approximately five “24 hour” time points—1,000 embryos per time point). Proteins are extracted and estimated, as described above, and stored until use.

Gel Electrophoresis and Western Blotting: For gel electrophoresis, 50 μg total protein is denatured in sample buffer (5× concentration: 0.3 M Tris-HCl, 5% SDS, 50%. glycerol, tracking dye, pH 6.8) by boiling for ˜5 minutes and run on a 10% SDS-polyacrylamide according to standard methods. Protein bands are identified by Comassie blue staining.

Following SDS-PAGE separation, proteins are transferred onto a 0.22 μm nitrocellulose membrane (BIO-RAD, California) using the mini Trans-blot mini cell protein transfer system (BIO-RAD, California). The nitrocellulose membrane is blocked using a power block solution (Pierce, Ill.) for 10 minutes at room temperature and subsequently washed in PBS-T (Phosphate buffered Saline pH 7.4 (PBS) containing 0.05% Tween-20 (Sigma, Mo.)) and incubated for 2 hours at room temperature with undiluted hybridoma culture supernatants positive on whole mount immunostaining. Unbound antibody is washed using PBS-T and the membrane is incubated with goat-anti mouse IgG conjugated to horseradish peroxidase (Pierce, Ill.) at a dilution of 1:2000 for 45 minutes at room temperature. Following incubation, the membrane is washed briefly with PBS-T followed by an extensive washing in PBS. The bound antibody is visualized using the Immunopure metal enhanced Diamino benzedene (DAB, Pierce, Ill.) according to manufacturer's instructions.

4. Isolation, Identifiction, and Characterization of Target Antigens

The relevant mAbs generated as described above are scaled up and purified by ascites production and immunoglobulin purification and is used to further characterize target proteins by immuno-histochemical staining and electron microscopic staining of zebrafish tissues. The purified mAbs are also used to isolate target antigens by standard immuno-precipitation methods (Harlow and Lane (1988), supra). Purified antigens are identified by sequence analysis using MALDI-TOF (Harvard Microchemistry Facility, Massachusetts) and used to identify (a) novel organ-, tissue-, or cell-specific markers and developmentally regulated markers and (b) mammalian homologues identified in protein databases. Sequences are also run on standard bioinformatic and proteomic databases to identify signature sequences that may reveal the functional properties of the target proteins. Finally, interesting liver-specific markers are used to develop organ-specific drug screening assays or to identify drug targets.

### EXAMPLE 3

Generation of a Liver-specific mAb

Using the methods set forth in Example 2, supra, liver tissue was isolated and used as an immunogen to generate a liver-specific IgG mAb, which was identified by screening whole mount zebrafish embryos. Staining of zebrafish liver with the liver-specific mAb and an fluorescently-labeled anti-IgG secondary antibody is shown in FIG. 1.

### EXAMPLE 4

In Vivo Ocular Neovascularization Screening Assay Using Eye Vessel-Specific mAb Phy-V002 to Identify Inhibitors of Angiogenesis

Conventionally, hypoxia induction has been generated by subjecting cells to a low oxygen atmosphere; but, for animals, long term exposure in a low oxygen atmosphere is not possible. Artificial hypoxic conditions have been created by initially exposing animals to a high oxygen atmosphere and then returning to a normal oxygen level. (See Shih et al., supra). However, this type of treatment must be performed over several days in order to condition the animal. Research has shown that zebrafish embryos at an early developmental stage (<24 hours post fertilization-hpf) can survive in anoxic or hypoxic atmosphere for 24 hours. (See Padilla and Roth, Proc. Natl. Acad. Sci. USA 98:7331-5, 2001; Ton et al., Physiol. Genomics 13:97-106, 2003.)

Hypoxia is thought to develop when the oxygen supply from the vasculature is diminished. Vasculature oxygen level is related to extracellular iron levels and heme protein synthesis and can be modulated by iron chelators. Several divalent transition metals such as Co⁺², Ni⁺², and carbon monoxide are known iron chelators, and were found to replace iron in the heme proteins, thus locking heme protein in its deoxygenated conformation and reducing the vasculature oxygen supply. Among the divalent transition metals, Co⁺², provided by CoCl₂, has been found to be a potent iron chelator. (See Rafii et al., Am. J. Physiol. Lung Cell. Mol. Physiol. 278:L399-L406, 2000.) CoCl₂ has been shown to induce HIF-1α expression and inhibits HIF-1α ubiquitination in human cells (see Jiang et al., J. Biol. Chem. 272:19253-60, 1997; Yuan et al., J. Biol. Chem. 278:15911-6, 2003); it has also been shown to increase the activity of HIF-1α transcriptional domain and up-regulate VEGF. CoCl₂ has been used as a hypoxia mimetic in a cell culture system. (See Ema et al., EMBO J. 18:1905-14, 1999; Sultana et al., J. Cell. Physiol. 179:67-78, 1999; Yuan et al., supra.)

Current methods of inducing choroidal neovascularization include subretinal injection of growth factors, laser treatment to disrupt retinal pigment epithelium complex (RPE), or a transgenic mouse model to overexpress VEGF by RPE (Schwesinger et al., supra, 2001). For the corneal neovascularization model, hydron pellets containing growth factors are implanted into the stromal layer near temporal limbus (Kenyon et al., Investigative Ophthalmology & Visual Science 37:1625-32, 1996). These methods rely on well-trained personnel to perform the surgical procedure, and usually take more than one week to determine if there is new vessel growth. As a model for retinopathy in premature infants, newborn mice are first placed in an atmosphere containing a high oxygen level and then brought back to a normal atmosphere to induce new vessel growth in the retina (Shih et al., supra). For a model of diabetic retinopathy, a daily injection of streptozotocin is often required to induce retinal neovascularization (Hammes et al., Nat. Med. 9:294-299, 2003). All these procedures require lengthy preparation and can only produce a few animals for drug treatment, which hinders the speed of drug discovery and limits an understanding of the mechanism of ocular neovascularization. Therefore, alternative, less invasive animal models for studying the process of ocular neovascularization and assessing drug effects are needed to facilitate identification of new therapeutics.

1. Development of a Hypoxia-induced Zebrafish Ocular Neovascularization Model: Characterize the Formation of New Blood Vessels in the Eye Using Whole Mount Immunostaining and Immunohistochemistry

A hypoxia-induced zebrafish ocular neovascularization model facilitates drug discovery in treating this serious condition. The structures and development of human and zebrafish eyes are highly conserved. The first vasculature to form in both species is very similar; and the histological structure of the retina is essentially indistinguishable in the two species. The laminar arrangement of the various cell types in the retina consists of orderly rows of cells separated by layers of their synaptic processes.

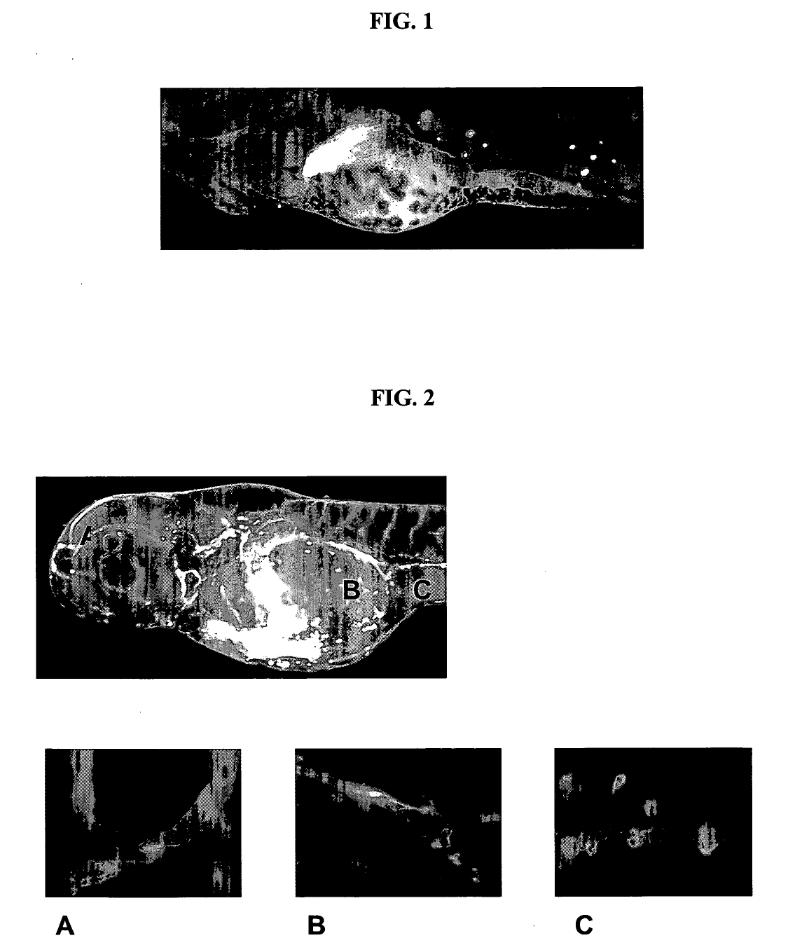

For the in vivo screening assay described in the this example, ocular neovascularization is induced in zebrafish by exposure to hypoxic conditions. Further, Phy-V002 mAb, specific for endothelial cells in the nascent vasculature, is used to visualize blood vessel formation. As shown in FIG. 2, individual cells forming the eye vessels (A), and sub-intestinal vessels (B and C) are clearly stained in a 2 dpf embryo by Phy-V002 mAb.

Zebrafish embryos at the 6 to 24 hpf stage are used for the ocular neovascularization model. Older embryos (>25 hpf) tend to be more sensitive to hypoxic stress and quickly died in hypoxic atmosphere (Padilla and Roth, Proc. Natl. Acad. Sci. USA 98:7331-7335, 2001). Embryos are generated as described in Example 1 (see also Westerfield, supra) and exposed to hypoxic conditions to induce neovascularization. Control and treated embryos at 1, 2, 3, 4 and 5 dpf are fixed and processed for whole mount immunostaining and examined by fluorescence microscopy.

Whole mount immunostaining: Embryos are fixed by Dent's fixative (DMSO/methanol=1/3) at room temperature (23±2° C.) for 3 hours, then dehydrated to 100% methanol following standard dehydration procedures. Embryos are then permeabilized, rehydrated, treated in 1 M NH4Cl at room temperature for 3 hours to quench autofluorescence, blocked, and incubated with the primary antibody following a standard procedure (Westerfield, supra). After washing with PBST, embryos are incubated with rhodamine-conjugated secondary antibody at room temperature for 2 hours. After washing, vessels are examined and photographed using an epifluorescence microscope.

Fluorescence microscopy: Fluorescence microscopy is performed using a Zeiss M2Bio fluorescence microscope (Carl Zeiss Microimaging Inc., Thornwood, N.Y.) equipped with a rhodamine cube and a green FITC filter (excitation: 488 nm, emission: 515 nm), and a chilled CCD camera (Axiocam MRM, Carl Zeiss Microimaging Inc., Thornwood, N.Y.). Screens are routinely done using a 1.6×, 10× and 20× objective archromats and 10× eye pieces. Additionally, a system equipped with a z-motorized stage, deconvolution software and 4-D reconstruction software (Axiovision, Carl Zeiss Microimaging Inc., Thornwood, N.Y.), which permits reconstruction of 3-D objects and analysis of Z-stacks, is used. Images are analyzed with Axiovision software Rel 4.0 (Carl Zeiss Microimaging Inc., Thornwood, N.Y.), Adobe Photoshop 6.0 (Adobe, San Jose, Calif.) and NIH image software (Bethesda, Md.).

Hypoxic conditions (e.g., time of exposure or level of oxygen deprivation) and starting embryo stage can be adjusted based on initial results. The condition that requires the shortest treatment time to consistently generate new vessels is used as the standard procedure for generating hypoxia-induced ocular neovascularization in zebrafish.

Immunohistochemistry: Representative embryos are processed for immunohistochemistry and examined by high resolution light microscopy to determine if new blood vessels penetrate into a deeper layer of tissues, such as corneal stroma, retina or vitreous body, which indicates a specific type of ocular neovascularization. Immunohistochemical analysis is performed as follows: embryos are fixed in paraformaldehyde and dehydrated in ethanol. Embryos are embedded in paraffin and 3-5 μm serial sections are generated. Sections are deparaffined, stained by hematoxylin following standard protocols, blocked, incubated with primary antibody, washed, and stained by rhodamine-conjugated secondary antibody. Sections are then mounted and examined by fluorescence microscopy.

Confirmation of hypoxia in treated eyes: Using whole mount immunostaining technique and Hypoxyprobe-1 (CHEMICON International Inc, Temecula, Calif.), a stain based on selective binding of 2-nitroimidazole (a hypoxia marker), the tissue oxygen gradient in control and treated embryos are examined and compared to confirm the hypoxia mechanism in new blood vessel formation in this model.

2. Confirmation of Up-Regulation of Hypoxia Induced Genes by RT-PCR and Identification of the Expression Site by in Situ Hybridization

The major gene induced in hypoxia is HIF-1, an oxygen-regulated transcriptional activator. HIF-1 binds to the hypoxia-response element located at the promoter region of the corresponding genes, thus up-regulating the expression of these genes (Semenza, Genes & Development 14:1983-1991, 2000). Some of these genes are involved in glucose/energy metabolism and some are related to vascular development and remodeling. To confirm that ocular neovascularization is hypoxia-induced, the expression levels of some of these representative hypoxia-induced genes (HIF-1α, PGK-1, VEGF, IGF) are examined to determine if they are up-regulated in hypoxia-treated embryos (Table 1). Also, as PEDF is a natural anti-angiogenic factor produced by pigment epithelial cells in the retina, expression of PEDF is examined to determine if this factor is down regulated. In addition, a house keeping gene, Tubulin α-2 cDNA, is included as a positive control.

TABLE 1
Representative genes affected by hypoxia.

| Genes | Accession ID | References |
|---|---|---|
| Hyperoxia inducible factor 1α | BC044475 | Semenza, supra |
| (HIF-1α) | | |
| 3-Phosphoglycerate kinase-1 | AAH46026 | Lay et al., Nature 408: |
| (PGK-1) | | 869-873 (2000) |
| Vascular endothelial growth | NM_131408 | Semenza, supra |
| factor (VEGF) | | |
| Insulin-like growth factor | AF250289 | Semenza, supra |
| (IGF) | | |
| Pigment epithelium derived | BQ285737 | Bouck, Trends in Mol. |
| factor (PEDF) | | Med 8: 330-334 (2002) |

RNA preparation: Based on results generated as described above, control and hypoxia-treated embryos are used to extract total RNA for RT-PCR analysis using specific primers based on the sequence identified from GenBank database in Table 1. Total RNAs from embryos is prepared by TRIzol reagent (GIBCO/BRL, Rockville, Md.). Briefly, 100 zebrafish embryos are homogenized and lysed in 1 ml of TRIzol reagent at 4° C. 200 μl of chloroform is added to the lysate, and the lysate is then incubated at 4° C. for 10 minutes or longer until phase separation occurs. The upper aqueous phase is transferred to a clean microcentrifuge tube. 600 μl of ice cold isopropanol is added, and centrifugation (12,000×g for 30 minutes) is used to precipitate RNAs.

A time chase experiment is used to investigate the gene expression profile; initially at 2, 4, 6, and 8 hours after exposure to hypoxic conditions, both control and hypoxia-treated embryos are collected and extracted for RT-PCR analysis. Based on the initial results, different time points may be required for sample collection, and the procedure is adapted and repeated at the appropriate time point. The PEDF expression profile is also determined.

Reverse transcription/Polymerase Chain Reaction (RT/PCR): Total RNA is reverse-transcribed with MMLV reverse-transcriptase (GIBCO/BRL) primed with oligo dT and subjected to PCR using gene-specific primers. PCR is performed using Platinum Taq (Invitrogen, Carlsbad, Calif.) in an MJ. Research (Reno, Nev.) PTC-100 thermocycler using the following cycling parameters: 25 cycles of (94° C. 30 sec, 68° C. 1 minute, 72° C. 1-2 minutes) followed by 72° C. 10 minutes. 12.5 μl of a 50 μl reaction is run on a 2.0% agarose gel to verify the expression level between control and hypoxia-treated embryos.

If needed to increase sensitivity for distinguishing expression of these neovascularization-related genes, as an alternative to RT-PCR, quantitative real time PCR is performed using the ABI 7700 sequence detection system.

To determine if changes in expression level are located at the site of ocular neovascularization, probes are generated using the same primers (Table 1) and in situ hybridization is performed in both control and hypoxia-treated embryos at the time point identified by RT-PCR results.

Probe labeling: To generate RNA probes for in situ hybridization, cDNA of genes of interest are generated by standard polymerase chain reaction (PCR) techniques using custom-designed primers. The cDNA is used as template to synthesize digoxigenin-labeled RNA probes, according to the manufacturer's protocol (Roche Diagnostics, Inc., Indianapolis, Ind.).

In situ hybridization: In situ hybridization is carried out as follows: embryos are fixed with 4% paraformaldehyde in PBS and re-hydrated with PBST. The RNA probes are hybridized at 65° C. in hybridization solution (50% formamide, 5× SSC, 0.1% tween 20, 0.05 mg/ml Heparin, 0.5 mg/ml tRNA, 10 mM Sodium Citrate buffer pH 6.0). Alkaline phosphatase-conjugated, anti-digoxigenin antibody is used to detect signals. For staining, embryos are equilibrated in NTMT buffer (0.1 M Tris/HCl pH 9.5, 50 mM MgCl₂, 0.1 M NaCl, 0.1% tween 20) at room temperature. Once the embryos have equilibrated, 4.5 μl of 75 mg/ml NBT (Nitro Blue Tetrazolium) and 3.5 μl of 50 mg/ml BCIP (5-Bromo-4-Chloro-3-Indolyl-Phosphate) per ml are added to the staining solution. The staining reaction is stopped by washing the embryos with PBST. Embryos are then examined on a stereo-dissecting microscope.

3. Evaluation of Inhibitory Effect of Antiangiogenic Compounds on New Blood Vessel Formation in Zebrafish and Comparison of the Results With Other Mammalian Models

Since the corneal pocket neovascularization model was originally used to test the anti-angiogenic effect of compounds, many anti-angiogenic drugs were first shown to be effective in inhibiting corneal neovascularization. The therapeutic focus has shifted to include the use of these agents to treat ocular neovascular diseases locally. Several drugs are currently undergoing preclinical or clinical evaluation as treatment options for diabetic retinopathy or macular degeneration. Some of these compounds are assessed for inhibition of neovascularization in embryo eyes after hypoxia treatment. Table 2 lists the compounds that are used.

TABLE 2
Test compounds for inhibiting ocular neovascularization
in hypoxia-treated zebrafish embryos

| Compounds | Mechanism | Source | Status |
|---|---|---|---|
| Radicicol | COX-2 | Calbiochem, | Another COX-2 |
| | inhibitor | La Jolla, CA | inhibitor, Celebrex, |
| | | | is currently in Phase II |
| | | | clinical trials for |
| | | | ARMD, sponsored |
| | | | by NEI |
| 2-methoxyestradiol | Inhibits cell | Calbiochem, | Under preclinical |
| | proliferation | La Jolla, CA | development for |
| | and | | treating ARMD |
| | angiogenesis | | by Allergan |
| | | | and EntreMed |

Drug treatment: Various concentrations of test compounds are dissolved in DMSO, added to the fish water, and incubated with 24 hpf hypoxia-treated embryos continuously. Hypoxia-treated embryos incubated with embryo water and 1% DMSO only are used as negative controls. At 2, 3, 4, and 5 dpf, embryos are collected and processed for whole mount immunostaining with Phy-V002; eye vessels are examined by fluorescence microscopy. Images of eye vessels are recorded and analyzed as described previously in the methods section for fluorescence microscopy for comparison between drug-treated and control embryos. Using data from published reports, results of these two drugs in the zebrafish ocular neovascularization model are compared with the results using other, mammalian eye neovascularization models.

### EXAMPLE 5

Quantitative In Vivo Microplate Screening Assay for Inhibitors of Angiogenesis Using Phy-V001 mAb

Microangiography is the most frequently used technique to assess zebrafish vessels (FIG. 3B). However, this technique requires a skilled technician to perform precise microinjection, and a functioning heart to pump the fluorescent microspheres through the circulatory system. Furthermore, all the vessels with open lumens in the embryo, including vasculogenic and angiogenic vessels, are stained. During embryogenesis, budding vessels often do not have open lumens, and, therefore, cannot be stained by this technique. Additional disadvantages of microangiography include that the technique cannot be accurately quantified, and drugs that affect heart function, but do not inhibit vessel formation, can be scored as false positives. Therefore, a rapid, specific quantitative assay that can be used to analyze angiogenic vessels is needed.

Materials and Methods

Chemicals and reagents: Chemicals and reagents were purchased from Sigma (St. Louis, Mo.). Rhodamine conjugated secondary antibodies were purchased from Jackson ImmunoResearch Laboratories, Inc. (West Grove, Pa.). Horseradish peroxidase (HRP) suppressor, HRP-conjugated secondary antibodies, 3,3′-diaminobenzidine concentrate (DAB) and QuantaBlu kit were purchased from Pierce (Rockford, Ill.). Coomasie Blue R-250, and Kaleidoscope Protein Standards were purchased from Bio-Rad (Hercules, Calif.). Hematoxylin was purchased from Zymed Laboratories (South San Francisco, Calif.).

Instrumentation: A fluorescence microscope with a CCD camera (Hamamatsu, Hamamatsu City, Japan), a SPECTRAmax Gemini microplate reader including software (Molecular Devices, Sunnyvale, Calif.), and a gel and Western blot apparatus (Bio-Rad) were used.

Embryo collection: Embryos were generated as described in Example 1 (see also Westerfield, supra).

Zebrafish vessels used as immunogen: For use as immunogen, we extracted vessels from 3-month old embryos. To expose the vessels, which were easily identifiable by their natural reddish black color using microscopy, zebrafish were dissected longitudinally from the abdominal side to the vertebra. Using fine tipped forceps, vessels were lifted and separated from the vertebra and immediately stored in phosphate buffered saline (PBS). To eliminate large debris, vessels from ˜50 embryos were pooled together, homogenized, and centrifuged at ˜1,000 rpm for 2 minutes. The supernatant (˜0.8 ml) was used as immunogen.

Whole mount immunostaining: Embryos were fixed by Dent's fixative (DMSO/methanol=1/3) at room temperature (23+2° C.) for 3 hours, dehydrated to 100% EtOH following standard dehydration procedures. Embryos were then permeabilized, rehydrated, treated in 1 M NH₄Cl at room temperature for 3 hours to quench autofluorescence, blocked, and then incubated with the primary antibody following a standard procedure (Westerfield, supra). After washing with PBST, embryos were incubated with rhodamine conjugated secondary antibody at room temperature for 2 hours. After washing, embryos were examined by fluorescence microscopy.

Immunization: Mouse immunization was performed by Cocalico Biologicals, Inc. (Reamstown, Pa.). Pre-immunization and post-immunization serum were collected and screened against zebrafish using the whole mount immunostaining technique described above. Only mice with positive serum were used to generate hybridomas.

Hybridoma production: Hybridoma production was performed using the standard polyethylene (PEG) fusion technique. The resulting hybridomas were plated into 96-well plates to be selected and grown (Harlow and Lane (1988), supra). Supernatant of the clones were tested using the fast whole mount immunostaining hybridoma screening technique described below. Positive wells were further diluted and plated to obtain single-cell clones.

Hybridoma screening using fast whole mount immunostaining: Embryos were treated as in whole mount immunostaining, as described above, up to the blocking step, and then distributed into a 96-well MultiScreen filtration plate (Millipore), 4 embryos per well. Embryos were then incubated with supernatant from the hybridoma clones at room temperature for 4 hours; control embryos incubated with blocking buffer without the supernatant were set up in the same plate. Using a vacuum manifold, unbound antibody was removed, and embryos were washed with PBST. Rhodamine-conjugated secondary antibody was then used to stain the embryos, again using the vacuum manifold to remove unbound antibody. Embryos in each well were examined directly using epifluorescence microscopy, and images were captured by a CCD camera. Using this semi-automated process, thousands of hybridoma clones can be screened in 2.5 days.

Zebrafish protein extract: To produce zebrafish protein extract, ˜3,000 embryos (3 dpf) were killed instantly by adding ice cold NP-40 lysis buffer (150 mM NaCl, 1.0% NP-40, 50 mM Tris, pH 8.0) (Harlow and Lane (1988), supra). Embryos were then homogenized by sonication (Branson Ultrasonics Corp, Danbury, Conn.) at 4° C. to extract proteins. Homogenates were centrifuged at 10,000 g for 10 minutes at 4° C. to precipitate large debris. The supernatant was used for immunoprecipitation.

Immunohistochemistry and microscopy: Immunohistochemical analysis was performed as follows: embryos were fixed in Dent's fixative and dehydrated in methanol. Embryos were embedded in paraffin and 3-5 μm serial sections were generated using a microtome. Sections were deparaffined, stained by hematoxylin following standard protocols, blocked, incubated with primary antibody, washed and stained by rhodamine-conjugated secondary antibody. Sections were then mounted and examined by fluorescence microscopy. Confocal microscopy was performed using Noran Odyssey XL Laser Scanning Confocal Microscope (Tufts University, Core Facility Confocal Microscopy Service, Boston, Mass.). Whole mount immunostained embryos were examined and images of 60 5 μm-slices were captured, compiled, and analyzed.

Microangiography: 3 dpf embryos were anesthetized with tricaine throughout injection and photography. Embryos were immobilized in holding troughs made of 1% agarose. A suspension of small (0.02 μm) red fluorescent (580/605) carboxylate-modified microspheres (Molecular Probes) was drawn into a pulled glass micropipette, which was attached to a picospritzer (General Valve), pressurized by nitrogen, and injected into the circulation of the 3 dpf embryo. Embryos were then examined using a fluorescence microscope. At least 20 embryos were used for each condition.

Drug treatment: Small molecule drugs, SU5416, SU9518 (SUGEN, Inc. (South San Francisco, Calif.), flavopiridol (NCI, Rockville, Md.), COL-1 and COL-3 (CollaGenex Pharmaceuticals, Inc., Newtown, Pa.) were used. 1 dpf embryos were treated with varying concentrations of each drug. At least 36 embryos were used for each concentration. Drugs dissolved in DMSO were added directly to fish water and diffused into the embryos. Embryos were incubated with drugs at 28° C.; the final concentration of DMSO was 0.1%. Embryos treated with 0.1% DMSO were used as drug carrier control. After drug treatment, embryos were washed and processed for each assay.

Quantitative in vivo microplate assay: Embryos were fixed, permeabilized, rehydrated, blocked, and incubated with primary mAb, as described in whole mount immunostaining, except that a horseradish peroxidase (HRP)-conjugated secondary antibody was used next instead of a rhodamine-conjugated secondary antibody. HRP suppressor (Pierce) was used to inhibit endogenous HRP. QuantaBlu (Pierce) was used as the fluorescence enzyme substrate, the vessel volume was quantified by measuring the fluorescence units of the enzymatic end product at 315-340 nm (excitation)/370-470 nm (emission) using SPECTRAmax Gemini microplate reader.

1. Assessment of EC Response to Angiogenic Inhibitors Using Zebrafish Specific Antibodies

To assess cross-reactivity in zebrafish, several commercially available polyclonal antibodies that target specific mammalian EC surface markers, including Flk1, Tie-1, Tie-2, CD31, and von Willebrand factor (Santa Cruz Biotechnology, Santa Cruz, Calif.) were first tested. Using 1-3 dpf embryos, faint staining and high background were observed with anti-CD31 and anti-FLK-1; no staining was observed with the other 3 antibodies. Since cross-reactivity was poor, these mammalian antibodies typically would not be useful reagents for an angiogenesis drug screen in zebrafish. Zebrafish vessel-specific mAbs were therefore generated for use in a rapid, quantitative in vivo zebrafish screen to identify anti-angiogenic drugs.

Characterization of zebrafish vessel specific mAbs: Using zebrafish vessel homogenate as immunogen, 14 hybridoma clones were generated. Using a rapid whole animal hybridoma screening technique, described supra, 6 clones specifically stained angiogenic vessels, including the intersegmental vessels (ISVs) and vessels in the head region, and did not stain the major vasculogenic vessels, including the dorsal aorta (DA) and posterior cardinal vessel (PCV) (FIG. 3A).

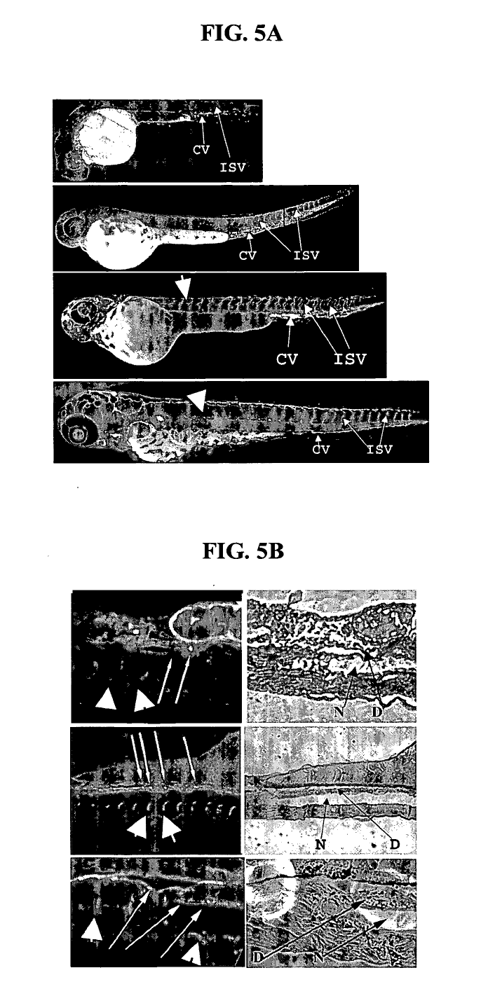

Zebrafish vessel development identified by Phy-V001: During early embryogenesis, the notochord, somites, and heart begin to form around ˜12 hpf, the DA and the PCV are known to form around ˜20 hpf. Using purified Phy-V001, the pattern of vessel development in different age embryos was also characterized. Phy-V001 staining was not detectable in 12 hpf embryos, but was detectable at ˜20 hpf, primarily in the caudal vein region (data not shown). The yolk sac was strongly autofluorescence (confirmed by DAB colorimetric staining, data not shown). At 24-28 hpf (1 dpf), ISVs and head region vessels were faintly stained. By 1.5 dpf, ISVs and head region vessels were strongly stained. As the embryos developed during 1.5-3 dpf, the complexity of the vessel pattern in the head region increased while the honeycomb structure of the caudal vein (CV) became elongated and less complex (FIG. 5A). Using microangiography, which detects the whole vasculture, identical changes in angiogenic vessel patterning have been observed (Isogai et al., Dev. Biol. 230:278-301, 2001). However, using Phy-V001 staining, the budding of the parachordal vessel (PAV) from ISVs was observed as early as 2 dpf, whereas using microangiography, the PAV was not detectable until ˜5 dpf (Isogai et al., supra). This difference is due to the fact that microangiography requires vessels to have open lumens to permit the flow of fluorescent microspheres; vessels at the budding stage often do not yet have opened lumen, and, therefore, they cannot be observed by microangiography. Further supporting the specificity of Phy-V001 for angiogenic vessels is that, throughout early embryogenesis (from 1-6 dpf), there was no evidence of staining in either the DA or the PCV. In contrast, these two major vasculogenic vessels were clearly stained by microangiography (FIG. 3B). These results support the conclusion that Phy-V001 is specific for angiogenic vessels.

To further confirm the specificity of Phy-V001 to angiogenic vessels and to eliminate the possibility of poor antibody penetration, immunohistochemical analysis on sectioned embryos was performed. Cross-sectioned embryo images showed that DA stained only at the site of ISV sprouting. Sagital-sectioned embryos showed that ISVs (large white arrowheads in FIG. 5B) were weakly stained in 1 dpf embryos and strongly stained in both 2 and 3 dpf embryos. However, the DA only positively stained at the site where ISVs sprouted (long white arrows in FIG. 5B), and weakly stained in other regions. Sections from older embryos (4, 5 and 6 dpf) showed strong staining in vessels in the gut, liver, and pancreas. It is well-recognized that vessels in the organs are primarily angiogenic vessels (Fouquet et al., Dev. Biol. 183:37-48, 1997; Zetter, Ann. Rev. Med. 49:407-24, 1998), further confirming the specificity of Phy-V001.

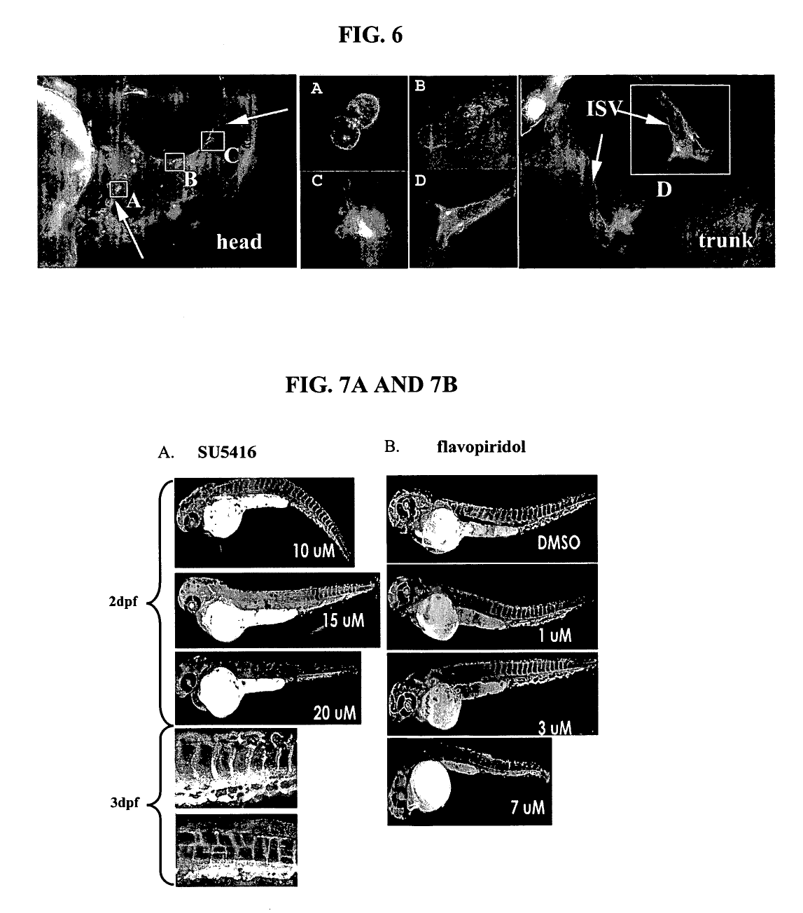

Phy-V001 mAb targets endothelial cells: Because the zebrafish embryo is transparent, using microscopy, single cell migration is observable during early embryogenesis (Childs et al. 2002). Using whole mount immunostaining with Phy-V001 in 40 hpf embryos, several vessels in the process of forming were observed (long white arrows in FIG. 6 head); the strongest staining appeared as several bright spots. At higher magnification (200×) (FIG. 6A, B, and C), these bright spots were identifiable as membranes on individual ECs. The strongest staining appeared at the adhesion (A), advancing (migration)/budding (proliferation) (B and C) sites of the ECs. In the trunk region, each ISV is comprised of three ECs: two T-shaped ECs, one on the dorsal end, one on the ventral end, and one EC form the connecting tube between the two T-shaped cells (Childs et al. 2002). These cells were stained by Phy-V001 (FIG. 6, trunk). The strongest staining appeared at the site where the parachordal vessel (PAV) branches from the ISVs (D). These results demonstrate that Phy-V001 stains activated ECs, especially in the regions where vessel sprouting occurs.

2. Development of a Whole Animal ELISA Assay to Measure the Effect of Drugs on Angiogenic Vessel Formation

Phy-V001 mAb is specific for angiogenic vessels and, therefore, can be used to detect drug effects on angiogenesis in zebrafish. This antibody was used to develop a quantitative in vivo microplate assay for measuring drug effects on the process of angiogenesis. To validate the assay and establish dose response curves for compounds, small molecules known to cause angiogenic effects in mammals were used.

Assay validation with small molecule drugs: Using the whole mount immunostaining technique described supra, several small molecule drugs were tested. SU5416 is an inhibitor of VEGF receptor, which is known to play a crucial role in EC response to angiogenic stimulators (Sundberg et al., Am. J. Path. 158:1145-60, 2001). SU9518 is an inhibitor of the PDGF receptor, which has been shown to assist in mobilizing smooth muscle cells to wrap around the endothelial cell tubule (Hellstrom et al., Development 126:3047-55, 1999). Flavopiridol is a cyclin-dependent kinase inhibitor known to inhibit cell proliferation (Senderowicz, Invest. New Drugs 17:313-20, 1999). COL-1 and COL-3 are inhibitors of matrix metalloproteases (MMPs), which are known to play a crucial role in vascular endothelial cell migration (Seftor et al. 2001). During early embryo development (˜12 hpf), many structures including the notochord, the somites, and the heart are beginning to form and these structures directly affect both vasculargenesis and angiogenesis. After 20 hpf, since the DA and the PCV are fully formed, drug effects on vessel formation are less likely to be secondary effects (indirect effects due to damage to the notochord, somites, or heart). Therefore, ˜1 dpf was chosed as the time point to initiate drug treatment. Embryos were incubated with varying concentrations of each drug continuously for 48 hours. Drug effects were examined at 2 dpf and 3 dpf.

a. Inhibition of Angiogenic Vessel Development Detected by Whole Mount Immunostaining

Inhibition of angiogenic vessel development detected by whole mount immunostaining. At 2 dpf As shown in FIG. 7A, after treatment with 10 μM SU5416 for 24 hours, the inhibitory effect was not obvious. However, when the concentration of SU5416 to 15 μM was increased, a dramatic decrease in Phy-V001 staining was observed; ISVs in the trunk region were affected first, followed by ISVs in the tail region. At 20 μM, ˜3% of the embryos died; the surviving embryos had truncated body formation and decreased staining in vessels in the nead region, including vessels in the eyes; ISVs were barely detectable. At 40 μM, ˜15% of the embryos died; the inhibitory effect on vessel development in surviving embryos was similar to effects using a 20 μM concentration of SU5416. In FIG. 7B, using a 1 μM concentration of flavopiridol, the inhibitory effect was not obvious; however, when the concentration of flavopiridol was increased to 3 μM, ISV staining in the trunk region decreased. Using a 5 μM concentration of flavopiridol, ˜16% embryos died; surviving embryos had truncated body formation, a dramatic decrease in ISV staining, and slightly decreased staining in vessels in the head region. At 7 μM, ˜20% embryos died; the surviving embryos had decreased staining in ISVs, similar to results in 5 μM treated embryos; staining in vessels in the head region vessels was undetectable.

In comparison, treatment with SU9518 using concentrations ranging from 0.1 μM to 100 μM did not cause a change in vessel development, compared to the control (data not shown). Treatment with COL-1 and COL-3 caused a loss of a few ISVs, but the change in vessel pattern was not as dramatic as the changes after treatment with SU5416 and flavopiridol (data not shown).

At 3 dpf. After treatment with 10 and 15 μM SU5416 for 48 hours, a few ISVs in the tail region were observed, but staining was weaker, the vessels appeared narrower, and the pattern of the ISVs was either disrupted or twisted; abnormal branching was also observed (FIG. 7A). Most of the 20 and 40 μM SU5416 treated embryos died at 3 dpf. After treatment with 1, 3, and 5 μM flavopiridol for 48 hours, ISVs in both trunk and tail regions were observed, again the staining intensity appeared weaker. All the 7 μM flavopiridol treated embryos died at 3 dpf. The vasculature of various concentrations of SU9518, COL-1, COL-3 treated embryos all appeared unchanged at 3 dpf compared to the controls.

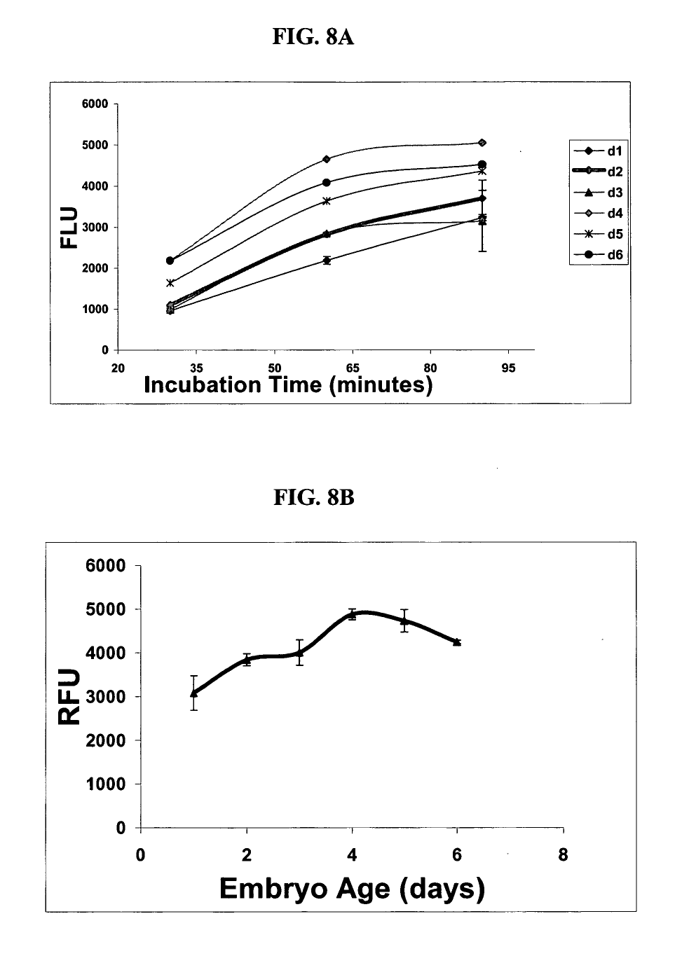

Establish optimal conditions for measuring angiogenic vessel development: In order to develop a reliable assay, optimal conditions to quantitatively distinguish different stages of vessel development were first establised. Normal 1 to 6 dpf zebrafish embryos with known vessel patterns were analyzed. As described supra, QuantaBlu was used as the soluble fluorescent HRP substrate. Since QuantaBlu is supplied in a fixed concentration, an optimal incubation time for the assay was first established. As shown in FIG. 8A, for most embryos, a linear relationship was observed during incubations ranging from 30 to 90 minutes. Optimal results were observed at 60 minutes; fluorescence units (RFU) were plotted against embryo age to confirm that 60 minutes was the optimum substrate incubation time for distinguishing the level of Phy-V001 staining for different age embryos (FIG. 8B). The increase in RFU in 4 dpf embryos correlated well with the increase in angiogenic vessels, primarily due to vessel growth in newly formed organs.

Calculation of Drug Effect: As discussed previously, level of fluorescence (FLU) from 1 dpf embryos was used as baseline. FLU of 0.1% DMSO treated embryos was used as 100% control. Drug Effect was expressed as % inhibition of angiogenic vessel development using the Formula (a):

$$\% Inhibition = ( 1 - \frac{FLU ( drug treated ) - FLU ( baseline )}{FLU ( Control ) - FLU ( baseline )} ) \times 100$$

As is typical with quantitative assays that rely on enzyme substrate, measurement of fluorescence units is affected by the batch of substrate produced by the manufacturer, which results in day-to-day and assay-to-assay fluctuations. In order to compare results from different assays performed on different days and reduce the contribution of substrate variability on results, the drug effect can be normalized to the % of inhibition in angiogenic vessel growth using Formula (a).

b. Dose Response Curves of Small Molecule Drugs

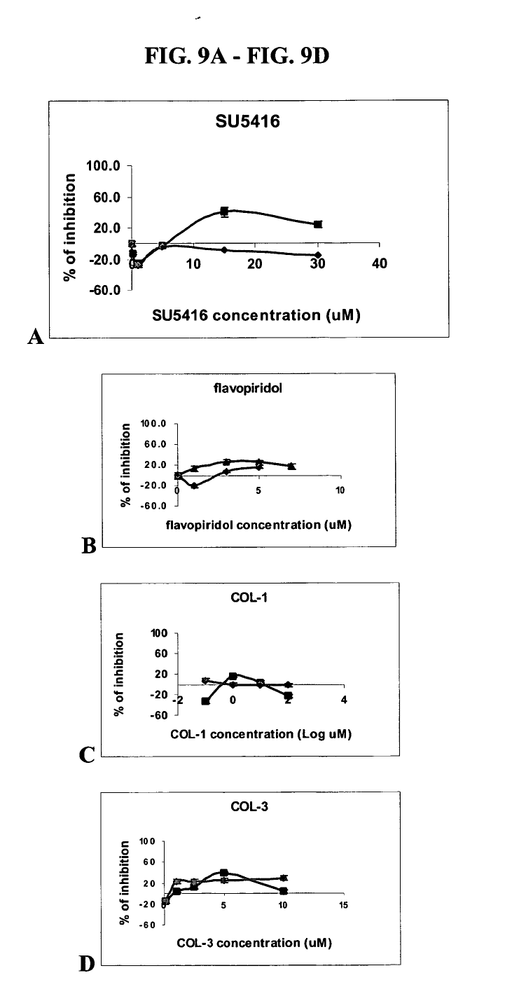

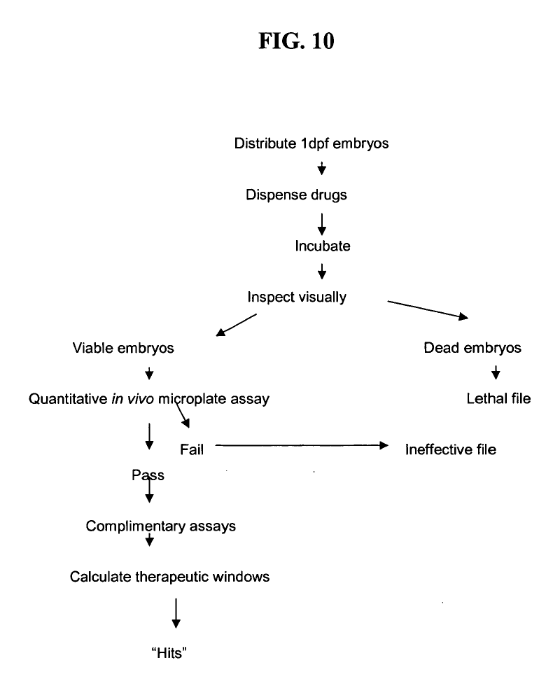

At 2 dpf: The dose response curves for 4 angiogenic compounds are shown in FIG. 9. Using a 15 μM concentration of SU5416, maximum inhibition in vessel growth was observed (˜40.5%, P<0.001). At concentrations higher than 20 μM, % of inhibition of angiogenic vessel growth did not increase (FIG. 9A). At a 3 μM concentration of flavopiridol, maximum inhibition of angiogenic vessel growth was observed (˜27%, P<0.002); at concentrations higher than 3 μM, % inhibition of angiogenic vessel growth did not increase (FIG. 9B). At a 1 μM concentration of COL-1, maximum inhibition of angiogenic vessel growth was observed (˜16%, P<0.5); at concentrations higher than 1 μM, no additional inhibition was observed (FIG. 9C). A 5 μM concentration of COL-3 caused the maximum inhibition of angiogenic vessel growth (˜39%, P<0.05) (FIG. 9D). In contrast, treatment with SU9518, in concentrations ranging from 0.1 μM to 100 μM, did not result in any angiogenic vessel inhibition. These results demonstrate that the maximum level of vessel inhibition and the optimum drug dosage differed significantly for the compounds tested.

At 3 dpf: No significant inhibition in angiogenic vessel growth was observed in the surviving embryos after 48 hours treatment with SU5416. Moderate inhibition was observed with 5 μM flavopiridol treatment (˜17%, P<0.05). No inhibition was observed with any of the COL-1 treated embryos. In contrast, 1 to 10 μM COL-3 treated embryos all showed significant inhibition on angiogenic vessel development (˜25%, P<0.0005).

Based on the above results, it was determined that 2 dpf is at least one optimum stage for assessing anti-angiogenic effects for several drugs.

Compare results with other models: Most anti-angiogenic drugs were first tested using either an in vitro EC proliferation assay or an in vivo mouse tumor model. Therefore, the results of the zebrafish in vivo model were compared with the results using these two assays (Table 3).

Results from the quantitative in vivo microplate assay correlated well with results using the mouse tumor model; the % vessel reduction was nearly identical. Although the 2-3 day assay time is similar to the time required for a cell-based assay, the mouse tumor assay requires 4 weeks. Additional advantages of the zebrafish model include: a single dosing regimen versus daily dosing for mouse, the ability to easily test statistically significant numbers of animals, and significantly lower quantities of drug for each test (2.5-5 ml/test for 50 zebrafish versus 10-20 ml/test for 6 mice), an important consideration for testing new therapeutics.

TABLE 3
Comparison of Drug Effects in Zebrafish, Mouse,
and EC Proliferation Assays

| | Zebrafish | Mouse Tumor¹ | EC Proliferation² |
|---|---|---|---|
| Assay Process Time | 2-3 days | 4 weeks | 3 days |
| SU5416 | | | |
| Vessel Reduction (%) | ˜40.5% | ˜42% | N.A. |
| Concentration | 2 μM | 12 mg/ml/day | N.A. |
| Dosing Frequency | 1 | 18 | N.A. |
| No. of animals/dose | 24-48 | 6 | N.A. |
| Flavopiridol | | | |
| Vessel Reduction (%) | ˜27% | N.A. | ˜32% |
| | | | (8-70% range) |
| Concentration | 3 μM | N.A. | N.A. |
| Dosing Frequency | 1 | N.A. | 1 |
| No. of animals/dose | 24-48 | N.A. | N.A. |

¹(Shaheen et al., Cancer Res. 59: 5412-16, 1999).
²Serum of patients treated with flavopiridol was used for this study (Murthy et al., ASCO Annual Meeting Abstracts, Abstract No. 1089, 2002)

Discussion

Whole mount immunostaining with Phy-V001 mAb is more sensitive than microangiography: By whole mount immunostaining using mAb Phy-V001 , ISVs as early as 1 dpf (FIG. 5A) were identified, and the complete set of ISVs was clearly stained by 1.5 dpf. The budding of the PAV was observed as early as 2 dpf. However, using microangiography, the earliest time point that ISV sprouting can be identified is 1.2-1.5 dpf, and the complete set of ISVs were only identifiable at ˜2 dpf. The PAV was not identified until 5 dpf (Isogai et al., supra). Using fli-GFP and tie-2-GFP transgenic zebrafish, the presence of ISVs was observed by 1 dpf by Lawson et al (Lawson and Weinstein, Dev. Biol. 248:307-18, 2002) and Childs et al. (Childs et al. 2002), in good agreement with our results using mAb Phy-V001 whole mount immunostaining.

A high throughput in vivo zebrafish angiogenesis assay was previosuly developed using endogenous alkaline phosphatase (EAP) as the marker (see, e.g., Seng et al., Keystone Symposia, Banf, Alberta, Canada: PA 253, 2002; Seng & McGrath, Drug Plus International 1:22-24, 2002). Although this assay format has been used to rapidly screen large compound libraries, it has several disadvantages for direct measurement of drug effects on angiogenic vessels, including: (1) the volume of the total vasculature is measured, and, therefore, vasulogenic vessels cannot be distinguished from angiogenic vessels, and (2) as evidenced by the intense staining in areas that have no blood vessels in both the head and the somite regions, this format has high background.

FIGS. 5 and 6 clearly demonstrate the specificity of Phy-V001 mAb for angiogenic vessels. In the head region, as embryogenesis progressed beyond ˜1.5 dpf, individual ECs in the process of forming vessels were observed. In the ISVs, budding/branching of ECs in the process of forming PAVs was also observed (FIG. 6 head and trunk). These results clearly indicate that Phy-V001 mAb stained activated ECs; expression of the target antigen was strongest in the budding (proliferation), or advancing (migration) region of the cells.

A major difference between whole mount Phy-V001 mAb staining and microangiography is that Phy-V001 antibody did not stain the heart and the major vasculogenic vessels (DA and PCV). This result indicates that only angiogenic vessels were stained by Phy-V001, underscoring the utility of Phy-V001 mAb as a reagent for distinguishing newly sprouted angiogenic vessels from vasculogenic vessels.

Correlation of drug effect by whole mount immunostaining with results using our quantitative in vivo microplate assay: Based on the results shown in FIG. 7, it appears that drug treatment affected ISVs in the trunk before ISVs in the tail. Using Phy-V001 staining, partial formation of ISVs was observed in 3 dpf embryos after treatment with SU5416 and flavopiridol, suggesting that the mechanism for inhibition of angiogenic growth by SU5416 and flavopiridol was partly due to delayed development, which was evidenced by total body truncation and deformation after drug treatment. COL-1 did not inhibit angiogenic vessel development as significantly as other drugs, and was completely ineffective at the 3 dpf stage. In contrast, COL-3 had significant inhibitory effect at both the 2 dpf and 3 dpf stages, delayed embryo development was also observed. SU9518 did not have any effect.

As shown in FIG. 9, the results of our quantitative assay correlated well with the visual results using whole mount immunostaining. For 1 μM flavopiridol treated embryos, 13% inhibition of angiogenic vessel growth was observed at 2 dpf, but visual results did not show significant difference from the control. For COL-3 treated embryos, our quantitative assay measured significant inhibition, but no significant changes in the vessel pattern was observed by whole mount immunostaining, except that the embryos appeared smaller. This result suggests that the quantitative assay is more sensitive than whole mount immunostaining, supporting the utility of the quantitative in vivo microplate assay for directly predicting the effects of drugs on angiogenic vessels. A significant advantage of the whole animal bioassay is that the therapeutic potential of a compound is evaluated in a complex physiological environment in a high throughput microplate format, similar to cell-based assays. Using cell-based screens, in vivo effects on vessel development are impossible to obtain.

### EXAMPLE 6

Development of Quantitative In Vivo Microplate Assay as a High Througput Angiogenesis Assay

1. Development of a High Throughput Quantitative in Vivo Microplate Bioassay and Conduct a Pilot Screen

The process for an automated quantitative in vivo microplate assay is as follows:
1) Distribute embryos, one embryo/well;
2) Drain fish water;
3) Add 100 μl drug containing fish water;
4) Incubate at 28° C. for 24 hours;
5) Drain drug; add 200 μl fish water to wash off the drug;
6) Drain fish water wash;
7) Add 100 ˜l Dent's fixative (DMSO:MetOH=1:3), and incubate for 2 hours at room temp;
8) Add 100 μl 100% MetOH and incubate for 10 minutes at room temp to dehydrate the embryos;
9) Drain above solution in step 8;
10) Add 100 μl HRP suppressor, and incubate for 15 minuttes at room temp;
11) Drain above solution;
12) Add 50 μl 100% MetOH, incubate for 10 minutes at room temp to permeabilize the embryo;
13) Add 50 μl PBST, incubate for 10 minutes;
14) Add 100 μl PBST, incubate for 10 minutes to re-hydrate the embryo;
15) Drain above solution from step 14;
16) Add 100 μl blocking solution; incubate for 1 hour at room temp;
17) Add 100 μl 2× primary mAb (Phy-V001), incubate for 2 hours at room temp;
18) Drain above solution from step 17;
19) Add 200 μl PBST; incubate for 10 minutes at room temp to wash off unbound antibody;
20) Drain solution from 19;
21) Add 200 μl PBS; incubate for 10 minutes at room temp;
22) Drain solution from step 21;
23) Repeat steps 20 and 21;
24) Add 100 μl HRP conjugated secondary mAb; incubate for 1.5 hours at room temp;
25) Drain solution from step 24;
26) Repeat steps 19 to 23;
27) Add 200 μl Tris buffer, pH 8.0, incubate for 10 minutes at room temp to equilibrate the embryo;
28) Drain solution from step 27;
29) Add 100 μl QuantaBlu; incubate for 60 minutes; and
30) Add 100 μl stopping solution, and measure fluorescence at 320/460 nm.

To perform a pilot screen, an automated liquid dispensing workstation MultiProbe II (Packard) is used to dispense reagents and solutions. Multiscreen filtration plates and a vacuum manifold are used to process zebrafish samples. 5 μM flavopiridol is used as the positive control drug, 5 μM SU9518 as the negative control, 1 dpf and DMSO treated 2 dpf (DMSOD2) embryos as the “0” and “100%” controls.

“Hit” Criteria and Database Management: Since it was observed that the fluorescence units (RFU) of DMSOD2 embryos can vary as much as ˜20%, in order to minimize false positive results, a stringent “hit” criteria is used: >20% vessel inhibition and P value of <0.05, compared to DMSOD2 controls. Based on visual observation immediately after drug treatment, a “lethal compound” file is set up for drugs that induced 100% death in embryos. Dead embryos are eliminated from data analysis. “Toxic compound” files are set up for compounds that have a low therapeutic window index, identified by subsequent complimentary assays. Using the ACCESS program, tables linking drug ID, dosage, and plate/well ID to the corresponding P value, % of vessel inhibition and the dose response curve are set up. Select queries based on the “hit” criteria are designed and used to identify a “hit” and stored in a file. Data on “hit” compounds is quickly retrieved for assessing further growth retardation, hemorrhage, and neuropathy assays.

Although throughput decreases using a multiple concentration test strategy, a dose response curve can be established, increasing assay accuracy. Using this approach, potential leads are identified early and compounds that are likely to fail in subsequent complimentary assays are rapidly eliminated. A significant advantage of the whole animal bioassay is that the therapeutic potential of a compound is evaluated in a complex physiological environment using throughput similar to cell based analysis. Using cell-based screens, this information is impossible to obtain.

2. Comparison of Anti-angiogenic Effects of “Hit” Drugs in Zebrafish and Mice

To confirm the anti-angiogenic effect of “hit” drugs identified by the zebrafish angiogensis screen, “hit” drugs are tested in the matrigel plug assay in mice, which is an established in vivo mammalian angiogenesis model. These experiments are performed by Paragon Bioservices, Inc., as described supra. In general, 10 mice are used for each condition, which includes 3 different doses of compounds, an injection vehicle, a negative control, and FGF positive control.

3. Development of Complimentary Assays to Assess Embryo Growth Inhibition Hemorrhage Edema, and Neuropathy after Drug Treatment

Recent reports of side effects of anti-angiogenic drugs during clinical trials support the need for a comprehensive drug-screening program comprised of a number of complimentary assays. In addition to inhibiting vessel growth, some drugs can affect overall embryo growth (see Example 5). Furthermore, some potential therapeutic compounds that do not produce immediate lethality can induce toxic effects in specific organs and tissues. Hemorrhage and neuropathy are the two major side effects observed in anti-angiogenic clinical trials. Assessment of drug effects on the nervous system and overall homeostasis is useful for predicting the extent of drug toxicity in order to establish the “therapeutic window.” Therefore, after identifying potential “hits”, the extent of growth retardation, hemorrhage, edema, and neuropathy are assessed.

Drugs cause growth retardation: Many drugs cause growth inhibition. Treatment with 5 μM flavopiridol for 4 days caused inhibition of overall embryo growth, determined by measuring body length. 1 dpf embryos are treated with identified “hit” drugs at various concentrations for 4 days. Body length is measured at 5 dpf and plotted against drug concentration. Normal untreated and 0.1% DMSO treated embryos are used as controls. At least 10 embryos are used for each condition.

Drugs cause hemorrhage and edema: After drug treatment, hemorrhage and edema often occur in zebrafish brain, yolk sac, heart, or trunk. If the heart is unable to maintain adequate blood flow throughout the circulatory system, excess fluid pressure within blood vessels shifts blood flow into the interstitial spaces giving rise to hemorrhage and edema. Some hemorrhage and edema caused by drug treatment was observed by dissecting microscopy. 1 dpf embryos are treated with “hit” drugs at various concentrations for 4 days; embryos are examined daily by dissecting microscopy. Blood pooling outside the circulation circuitry is defined as hemorrhage; abnormal expansion of organ structure (such as heart) is defined as edema.

For drugs that affect blood cell pigmentation, hemorrhage cannot be easily observed. Therefore, microangiography is performed to detect leaky vessels. Diffusion of fluorescent microspheres outside the circulation circuitry is defined as leaky vessels. Images are captured and analyzed, as described supra. At least 10 embryos are used for each condition.

Drugs cause neuropathy: Using dissecting microscopy, overall neurotoxicity of drugs is observable as necrosis in the head region. 1 dpf embryos are treated with various concentrations of “hit” drugs for 4 days, the embryos are examined daily, and images of brain necrosis are recorded. Again, at least 10 embryos are used for each condition.

Anti-mouse-acetyl-tubulin antibody, recognized as a neuronal cell marker for all species, can be used to identify differences in nerve development in the zebrafish brain. Since nerves develop earlier than other tissues in zebrafish, 2 hpf embryos are treated with various concentrations of “hit” drugs for 4 days. 10 embryos are recovered daily from each condition and whole mount immunostaining is performed using anti-acetyl-tubulin antibody. Stained embryos are examined using fluorescence microscopy, recorded, and analyzed, as described in Example 5.

Establish the therapeutic window (TW): The difference between the toxic concentration (TC) and the effective concentration (EC) determines the useful range for each drug: the wider the window, the safer the drug. Since the maximum angiogenic vessel inhibition observed by the present inventors using available compounds is less than 50%, the drug concentration that causes 30% inhibition as EC30 and the concentration of a drug that causes 50% toxic effect as TC50 are determined, and the ratio of TC50/EC30 is used as an index to compare the therapeutic window (TW). The higher the TW value, the less toxic the drug, and the higher the potential as a useful therapeutic.

### EXAMPLE 9

Whole zebrafish in Vivo EC Proliferation, Migration, and Vessel Formation Assays Using Phy-V002 mAb

Assay Protocols

Quantitative in vivo EC proliferation assay: Embryos were fixed by Dent's fixative (DMSO/methanol=1/3) at room temperature (23+2° C.) for 3 hours, dehydrated to 100% EtOH following standard dehydration procedures. Embryos were then permeabilized, rehydrated, treated with horseradish peroxidase (HRP) suppressor to inhibit endogenous HRP. After treatment with 1 M NH₄Cl at room temperature for 3 hours to quench autofluorescence, embryos were then blocked and incubated with Phy-V002 mAb following a standard procedure (see Westerfield, supra). After washing with PBST, embryos were incubated with HRP-conjugated secondary antibody. QuantaBlu (Pierce) was used as the fluorescence enzyme substrate; the stained EC was quantified by measuring the fluorescence units of the enzymatic end product at 315-340 nm (excitation)/370-470 nm (emission) using SPECTRAmax Gemini microplate reader (Molecular Devices).

Drug effect on EC proliferation was calculated by the following formula:

$$\% Inhibition = ( 1 - \frac{FLU ( drug treated )}{FLU ( Control )} ) \times 100$$

In vivo EC migration assay: Embryos were processed following the procedures described in “Quantitative in vivo EC proliferation assay,” supra, except rhodamine-conjugated secondary antibody was used to replace HRP-conjugated secondary antibody. After washing, embryos were examined by fluorescence microscopy. Presence of stained EC aggregates outside the vasculature was considered to be positive for inhibiting EC migration. Images of the control and drug treated embryos in the region where cell aggregation was observed were captured by CCD camera and analyzed by Adobe Photoshop Software (Auto FX Software, Birmingham, AL).

In vivo vesselformation assay: Embryos were processed as described in “In vivo EC migration Assay,” supra, and examined by fluorescence microscopy. Images of stained embryos were captured by CCD camera and analyzed by Adobe Photoshop software. Higher magnification (10×) images of control and drug treated embryos at the region where vessel defects were observed were quantitated by morphometric analysis (Signal (S)=[area (pixel) X signal intensity]) using Scion Image program. Percent (%) inhibition in vessel formation can be calculated by the following formula:

$$\% Inhibition = ( 1 - \frac{S ( drug treated )}{S ( Control )} ) \times 100$$

Results
1. Quantitative in Vivo EC Proliferation Assay

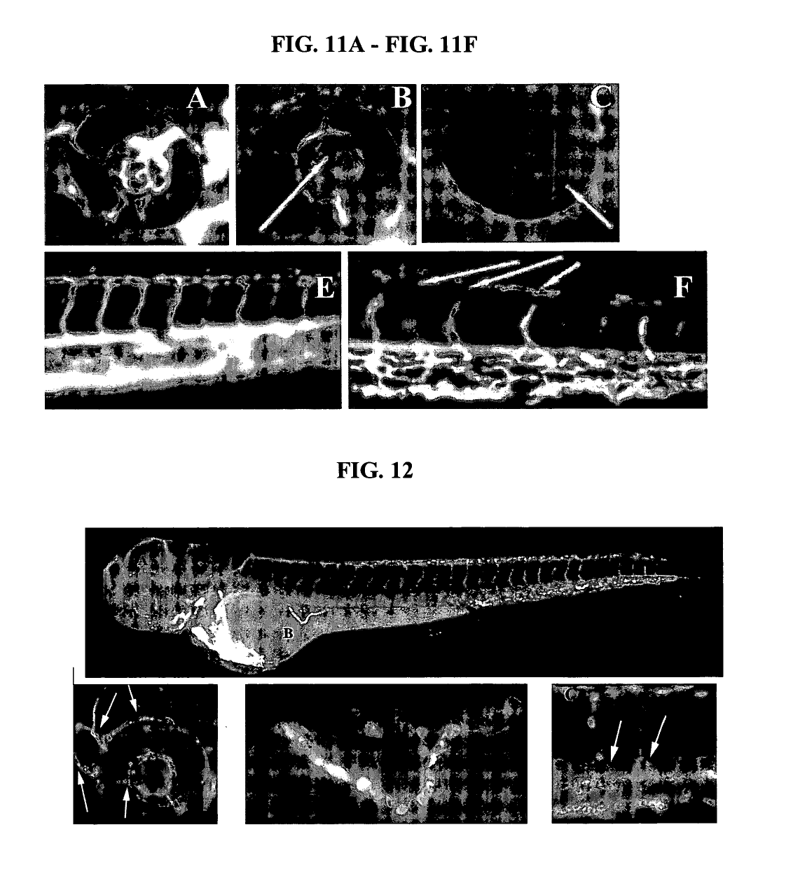

Phy-V002 mAb specifically stained activated vascular endothelial cells. Further, individual endothelial cells that are in the process of forming vessels can be distinguished with Phy-V002 mAb at higher magnification. (See FIG. 12, arrows.) This result demonstrated that the intensity of the staining correlates with the quantity of activated endothelial cells.

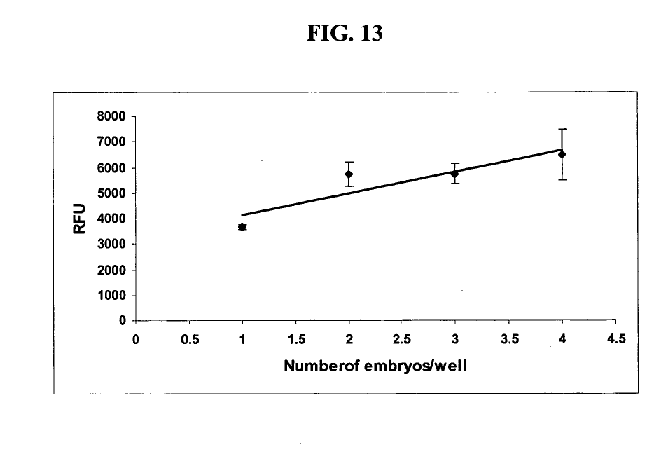

Based on the above observation, a whole animal Phy-V002 ELISA (as describe in Assay protocols, supra) was developed to measure the quantity of total activated endothelial cells in the zebrafish embryo. The linearity between the staining signal and the number of stained embryos (see FIG. 13) demonstrated the usefulness of the ELISA in quantitating in vivo EC proliferation in the whole embryo.

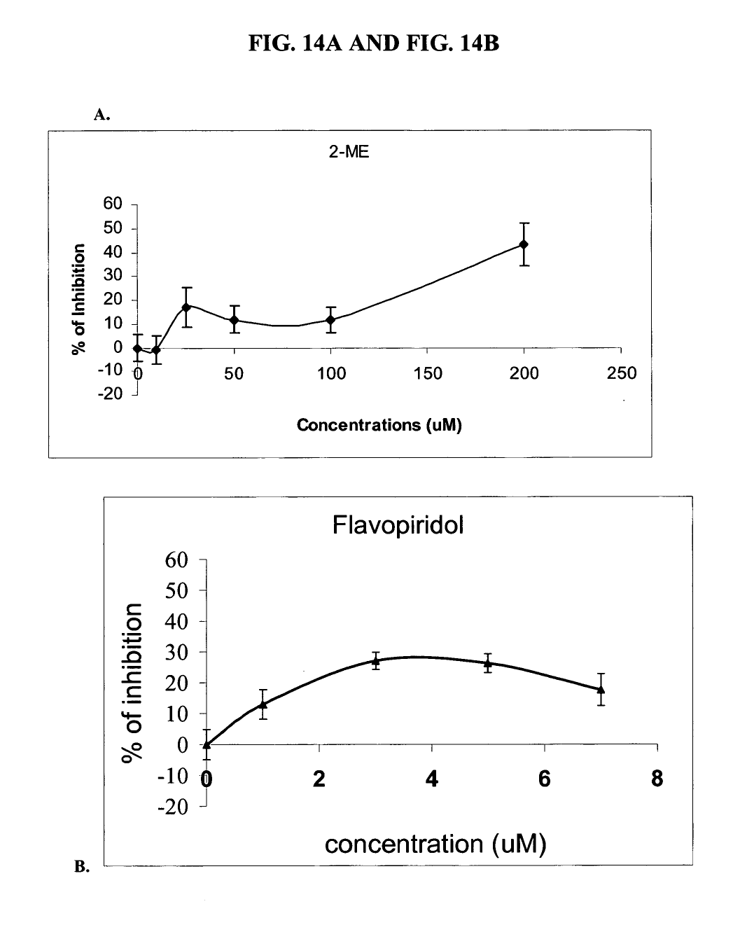

2-methoxyestradiol (2-ME) and flavopiridol, which have been shown to inhibit EC proliferation, were used to validate the in vivo EC proliferation assay. Typical dose response curves for were found for both drugs, with % inhibition in EC proliferation increasing proportionally with drug concentration. (See FIG. 14) These results validated the utility of the whole animal Phy-V002 ELISA as an in vivo EC proliferation assay.

2. Whole Zebrafish in Vivo EC Migration Assay

Angiogenic vessels form during normal tissue growth and repair in a series of sequential steps: endothelial cells (EC) (capillary) become activated, secrete enzymes that degrade the extracellular matrix (the surrounding) tissue, begin dividing, and invade the matrix. Eventually, strings of new endothelial cells organize into hollow tubes, creating new networks of blood vessels.

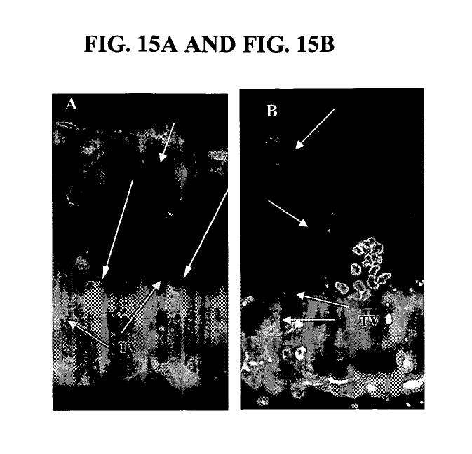

The enzymes that degrade extracellular matrix tissue are categorized into a large family of matrix metalloproteinases (MMPs). Several anti-angiogenic drugs were developed based on their anti-MMP mechanism and were shown to inhibit EC migration in the in vitro cell-based assay. Tetracycline, a collagenase inhibitor, was recently identified as a candidate drug in this category. Phy-V002-stained cells lined up clearly in well formed intersegmental vessels (ISV) and tail veins (TV) in the control embryo without any cells aggregated outside the vasculature. (See FIG. 15A, long white arrows.) In embryos treated with 600 μM tetracycline, ISVs were weakly stained and some part of ISVs were missing (see FIG. 15B, short white arrow), and Phy-V002 stained cells aggregating outside the vasculature was observed. TVs were abnormal. This result showed that Phy-V002-stained cells did not migrate to the proper site to form vessels in tetracycline treated embryos, and demonstrated that an inhibitory effect in EC migration can be detected in the whole zebrafish using whole mount Phy-V002 staining.

3. Whole Zebrafish in Vivo Vessel Formation Assay

As stated previously, the activated ECs, after proliferation and migration, eventually will line up properly to form a vessel; this process is usually assayed by in vitro cord formation assay, or in in vivo animal models. The in vitro assay does not provide physiological conditions; the available in vivo assays often require long drug treatment time, or special instrumentation and processing, rendering the assay time consuming and expensive.

The zebrafish in vivo angiogenesis assay can be conducted with 28 hours drug treatment; whole mount immunostaining does not require lengthy sectioning and immunohistochemistry procedures, and the embryo is completely transparent for easy observation.

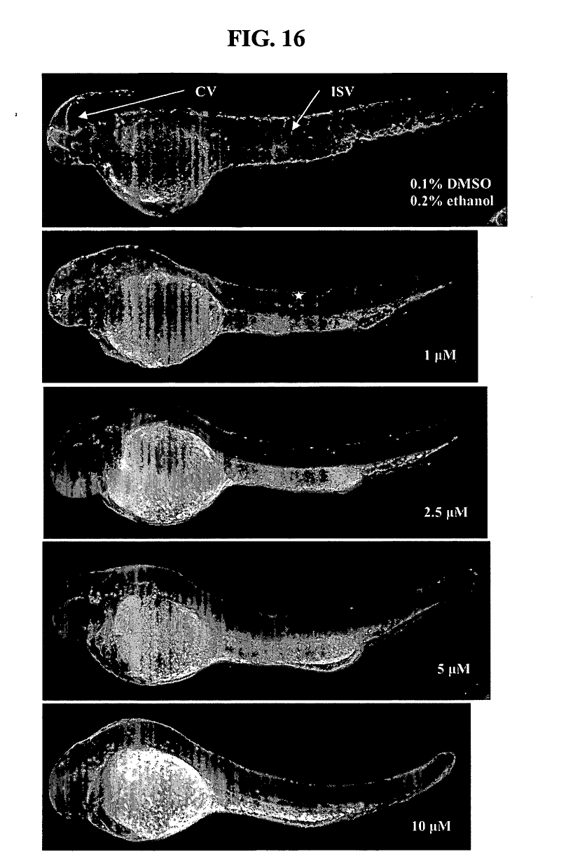

Although SU5416 (SUGEN) failed in clinical trials, primarily due to toxicity issue, it was a well known anti-angiogenic drug and, therefore, was used to validate the in vivo vessel formation assay. FIG. 16 shows inhibition of vessel formation by SU5416; vessels in the cranial region (CV) and the somite region (ISVs) were partially missing (stars) at 1 μM concentration, and were nearly completely absent at concentrations 2.5 μM and higher. This result demonstrated our Phy-V002 based zebrafish angiogenesis assay can easily detect inhibition in vivo of vessel formation.

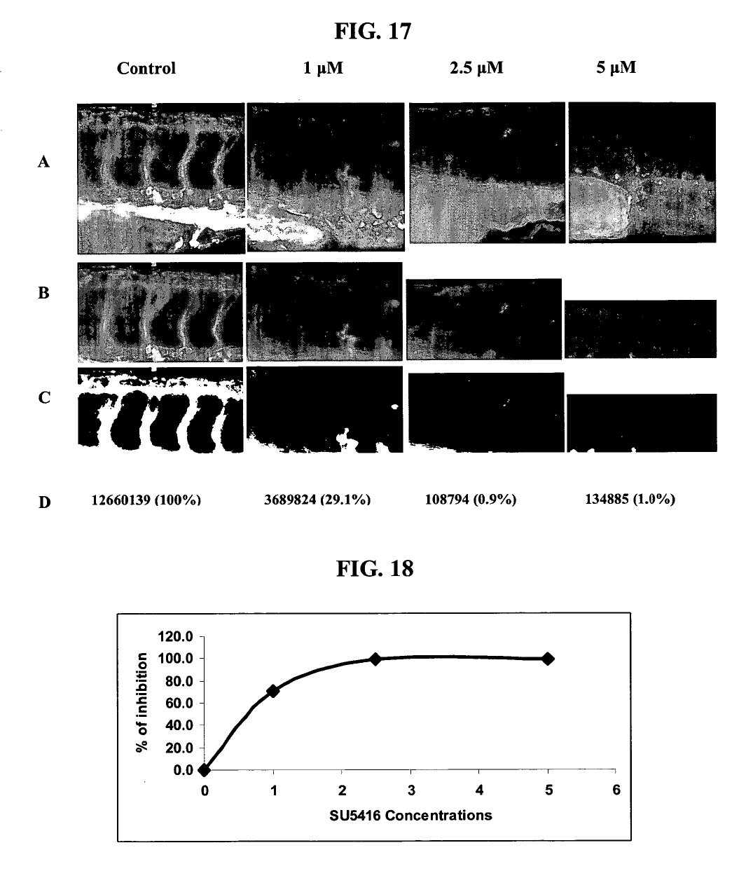

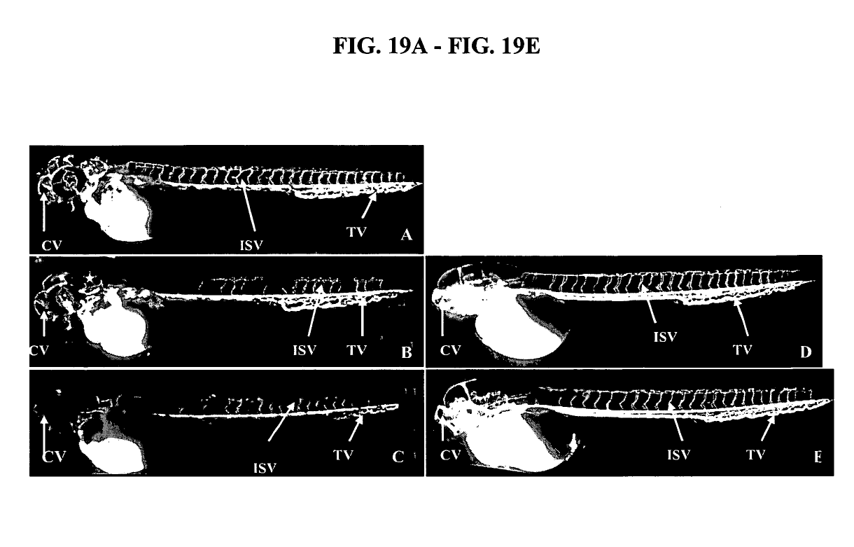

The same embryos were used to perform morphometric analysis to quantitate in vivo vessel formation. (See FIG. 17) The value obtained was then used to calculate the % inhibition and a dose response curve was generated by plotting the % inhibition against the concentrations used. (See FIG. 18) The results correlated well with visual observation, and validated the zebrafish in vivo vessel formation assay.

The previous examples are provided to illustrate but not to limit the scope of the claimed invention. Other variants of the inventions will be readily apparent to those of ordinary skill in the art and encompassed by the appended claims. All publications, patents, patent applications, and other references are hereby incorporated by reference in their entirety for all purposes.

1-32. (canceled)

33. A method of producing an organ-, tissue-, or cell-specific antibody, the method comprising:
(1) immunizing a mammal with cellular or extracellular material isolated from an animal of a species that is transparent through multiple stages of development to generate cells that secrete antibodies;
(2) contacting the antibodies with an intact animal of the transparent species, whereby the antibodies permeate the animal; and
(3) detecting the antibodies within the animal and thereby identifying an antibody that binds to the specific organ, tissue, or cell population.

34. The method of claim 33, further comprising isolating the cellular or extracellular material.

35. The method of claim 33, wherein the cellular or extracellular material comprises antigenic determinants from a specific organ or tissue selected from the group consisting of blood vessel, liver, pancreas, pharynx, esophagus, pronephros, brain, heart, gut, and muscle.

36. The method of claim 33, wherein the transparent animal is a teleost.

37. The method of claim 36, wherein the teleost is a zebrafish.

38. The method of claim 33, wherein the antigen recognized by the organ-, tissue-, or cell-specific antibody is developmentally regulated.

39. The method of claim 38, wherein the cellular or extracellular material is isolated from an animal that has a disease or has been treated with a drug.

40. The method of claim 39, wherein the intact animal of the transparent species has the disease or has been treated with the drug.

41. A method of producing an antibody to a developmentally regulated antigen, the method comprising:
(1) immunizing a mammal with cellular or extracellular material from an animal of a species that is transparent through multiple stages of development to generate cells that secrete antibodies;
(2) contacting samples of one of the antibodies with a plurality of intact animals of the transparent species, said intact animals representing at least two different stages of development, whereby the antibody permeates the intact animals;
(3) detecting the amounts and locations of the antibody specifically bound within the animals; and
(4) determining whether a difference in the amount and/or location of bound antibody is present between animals in different developmental stages, whereby if a difference in the amount and/or location of bound antibody is present, the antibody is identified as specifically binding to a developmentally regulated antigen.

42. The method of claim 41, further comprising isolating the cellular or extracellular material.

43. The method of claim 41, wherein the developmentally regulated antigen is organ-, tissue-, or cell-specific.

44. A method for identifying a protein for drug target validation, the method comprising:
(1) immunizing a mammal with cellular or extracellular material from an animal of a species that is transparent through multiple stages of development to generate cells that secrete antibodies, wherein the cellular or extracellular material comprises antigenic determinants from a specific organ, tissue, or cell population;
(2) contacting the antibodies with an intact animal of the transparent species, whereby the antibodies permeate the animal;
(3) detecting the antibodies within the animal and thereby identifying an antibody that binds to the specific organ, tissue, or cell population;
(4) isolating an antigen specifically bound by the antibody; and
(5) determining one or more biochemical characteristics of the isolated antigen to identify the protein for drug target validation.

45. The method of claim 44, wherein the biochemical characteristic is an amino acid sequence for the antigen or a fragment thereof.

46. The method of claim 44, wherein the biochemical characteristic is a mass spectral fingerprint.

47. The method of claim 46, wherein the mass spectral fingerprint is a MALDI-TOF mass spectral fingerprint.

48. The method of claim 44, further comprising identifying the gene that encodes the protein.

49. The method of claim 44, further comprising identifying a species variant of the protein or of the gene encoding the protein.
